(19) United States
(12) Patent Application Publication
(10) Pub. No.: US 2024/0043806 A1
(43) Pub. Date: Feb. 8, 2024

(54) MID-SCALE VESSELS IN ENGINEERED TISSUE FOR REGENERATION

(71) Applicant: Brown University, Providence, RI (US)

(72) Inventors: Kareen L. K. COULOMBE, Pawtucket, RI (US); Rajeev J. KANT, Providence, RI (US); Kiera D. DWYER, Providence, RI (US)

(21) Appl. No.: 18/358,808

(22) Filed: Jul. 25, 2023

Related U.S. Application Data
(60) Provisional application No. 63/369,338, filed on Jul. 25, 2022.

(51) Int. Cl. C12N 5/077

(52) U.S. Cl. CPC C12N 5/0657; C12N 2502/28; C12N 2502/45; C12N 2539/00

(57) ABSTRACT

The invention provides a dual remuscularization-revascularization therapy comprising vascularized engineered human myocardial tissues (vEHMs).

### CROSS-REFERENCE TO RELATED APPLICATIONS

This patent application claims priority under 35 U.S.C. § 119(e) to the provisional patent applications U.S. Ser. No. 63/369,338, filed Jul. 25, 2022.

### STATEMENT REGARDING FEDERALLY SPONSORED RESEARCH OR DEVELOPMENT

This invention was made with government support under grant numbers HL135091 and AG067228 awarded by the National Institutes of Health. The government has certain rights in the invention.

### TECHNICAL FIELD OF THE INVENTION

This invention generally relates to composite materials, i.e., containing materials for grafts or prostheses or for coating grafts or prostheses containing ingredients of undetermined constitution or reaction products thereof, e.g., transplant tissue, or extracellular matrix containing added animal cells.

### BACKGROUND OF THE INVENTION

Myocardial infarction (MI) interrupts vascular blood flow to downstream muscle, resulting in ischemia and necrosis of cardiac tissue. Restoration of cardiac contractility after myocardial infarction is important to prevent heart failure.

Heart regeneration after myocardial infarction (MI) using human stem cell-derived cardiomyocytes (CMs) is rapidly accelerating with large animal and human clinical trials. Vascularization methods to adequately support the engraftment, survival, and development of implanted cardiomyocytes in the harsh, ischemic environment of the infarcted heart remain a key and timely challenge.

There is an unmet need in the biomedical art to engineer a vascular supply for engineered human myocardium to ensure tissue survival and engraftment upon implantation to improve therapeutic efficacy.

### SUMMARY OF THE INVENTION

The invention provides a dual remuscularization-revascularization system. The inventors evaluated the dual remuscularization-revascularization system in a rat model of ischemia-reperfusion myocardial infarction. The system involves the differentiation of human induced pluripotent stem cell-derived cardiomyocytes (hiPSC-CMs) for engineering cardiac tissue containing patterned engineered vessels.

In a first embodiment, the invention provides a dual remuscularization-revascularization system comprising vascularized engineered human myocardial tissues (vEHMs) in a matrix biomaterial. The system comprises patterned mid-diameter vessel structures with branch points in a matrix biomaterial. The system also comprises endothelial cells (ECs) coated on the vessel structure. The vascular endothelial cells are localized to a vessel wall.

Cardiomyocytes alone or in mixtures with fibroblasts and/or vascular cells creates a solid 3D myocardial tissue. Sacrificially patterned biomaterials are embedded in myocardial tissues and coated with vascular endothelial cells.

Removal of the sacrificial material in physiological conditions amenable to the cells washes away the core biomaterial of the vessel pattern to leave hollow channels lined by endothelial cells in the tissue.

In a second embodiment, the invention provides that the vessels of the system, after implantation into a subject, self-connect to the subject's vasculature for greater perfusion. The customized patterning of vessels, localization of endothelial cells, and embedding with cells in hydrogels provides a therapeutic improvement.

In a third embodiment, the invention provides a method of making a dual remuscularization-revascularization system by constructing vascularized engineered human myocardial tissues (vEHMs) using a polydimethylsiloxane (PDMS)-based replica molding system for embedding biomaterials and cells. Vascularized engineered human myocardial tissues (vEHMs) are cultured in static conditions or perfused in vitro before implantation and evaluated after two weeks. Immunohistochemical staining indicated improved engraftment of hiPSC-cardiomyocytes in in vitro-perfused vascularized engineered human myocardial tissues with greater expression of SMA+ vessels and evidence of inosculation. Mid-diameter vessel structures with branch points are patterned using a sacrificial biomaterial such as alginate, sugars, etc. Fiber-based thread patterning or 3D bioprinting are compatible with this approach. Coating with endothelial cells on the vessel structure localizes these vascular cells to the vessel wall. Coating is done by dipping, painting, dripping, droplet pulling, or 3D bioprinting such as in a core-shell configuration. Embedding the vessel structure in a matrix at high densities creates a solid 3D tissue. The invention provides an arteriole-scale vessel fabrication and a channel endothelialization via ensheathment. In vitro perfused hiPSC-cardiomyocyte tissues with arteriole-scale, endothelial cell-lined vessels should help with greater perfusion of implanted tissues and improve hiPSC-cardiomyocytes survival and maturation in vivo.

Ex vivo optical coherence tomography shows less tortuous vascular organization in perfused vEHMs relative to non-perfused vEHMs. The 3D vascular reconstructions show less tortuous and larger intra-implant vessels, as well as improved branching hierarchy in in-vitro-perfused vascularized engineered human myocardial tissues relative to non-perfused controls.

In a fifth embodiment, the invention provides a method of using the system for therapeutic effect. The vEHM integration with hierarchical vascular perfusion and maximal hiPSC-cardiomyocyte engraftment enhances regenerative therapy. Such implanted tissues can be used in surgical settings by physicians to restore cardiac contractile function for patients with ischemic heart disease.

In a sixth embodiment, the invention provides a platform to assess the structural and functional effects of patterning arteriole-scale channels in engineered human myocardium.

In a seventh embodiment, the invention can be used to evaluate the ability of vascularized engineered human myocardial tissues to engraft, improve perfusion, and remuscularize the heart using a rodent model of ischemia/reperfusion myocardial infarction. This aspect shows the full in vitro to in vivo pipeline of developing the engineered human myocardium, patterning endothelialized vasculature, in vitro perfusion, implantation, and evaluation. The workflow shows the fabrication of stable engineered human myocardium with patterned, arteriole-scale vasculature that can successfully inosculate with host vasculature upon implantation and significantly improve tissue engraftment.

In an eighth embodiment, the invention provides bulk RNA-sequencing of explanted engineered human myocardium. Exploratory RNA sequencing of explanted vascularized engineered human myocardial tissues suggests that co-revascularization impacts hiPSC-cardiomyocyte development in vivo.

### BRIEF DESCRIPTION OF THE DRAWINGS

For illustration, some embodiments of the invention are shown in the drawings described below. Like numerals in the drawings indicate like elements throughout. The invention is not limited to the precise arrangements, dimensions, and instruments shown.

FIG. 1 is a diagram presenting an overview of the project to make patterned vessels, embed them into tissues, and evaluate the vEHMs in vivo.

FIGS. 2A-2B presents an overview of hiPSC-cardiomyocyte differentiation, vEHM formation, and in vivo implantation. FIG. 2A is a differentiation timeline of hiPSCs into cardiomyocytes. The inventors observed ventricular cardiomyocytes using multi-stage biphasic Wnt modulation. Differentiation purity was evaluated using flow cytometry staining for cTnT. FIG. 2B is an overview of timeline for in vivo implantation of engineered human myocardial tissues in a rat model of ischemia-reperfusion myocardial infarction.

FIG. 3A-3B is a set of graphs shows the mixed media culture differentially affects endothelial cell (EC) and cardiomyocyte structure and function. FIG. 3A is a line graph showing that endothelial cell proliferation and morphology evaluated in monolayer culture with mixed media. n=9 per group. The inventors observed compaction of 3D linear engineered human myocardial tissues across seven days of culture. FIG. 3B is a line graph showing the necking of 3D linear engineered human myocardial tissues across seven days of culture. n=18-21 per group. FIG. 3C is a bar graph showing the mechanical characterization of passive force generation of engineered human myocardial tissues at 5% stretch increments. FIG. 3D is a line graph and FIG. 3E is a bar graph showing the mechanical characterization of active stress generation of engineered human myocardial tissues at 5% stretch increments up to 30% stretch. n=18-21 samples per group. The inventors also observed histological staining of engineered human myocardial tissues using α-sarcomeric actinin (α-SA) and Connexin-43 (Cx43) after mechanical characterization.

FIGS. 4A-4B show the formation of patterned vasculature using EC-coated gelatin-alginate fibers. FIG. 4A is a bar graph showing equilibrium swelling and diameter assessment of gelatin-alginate fibers in mixed media culture. n=9 per group. Significance relative to 100/0 medium of the same fiber composition is shown. FIG. 4B is a bar graph showing solutions composed of gelatin, alginate, collagen, or a combination thereof are used to evaluate coating smoothness for homogenous endothelial cell seeding. n=3 per group. The inventors observed an endothelial cell-coated vessel using gelatin-alginate-collagen solution in whole mount and transverse sections.

FIGS. 5A-5C are graphs showing fabrication and perfusion culture of vascularized engineered human myocardial tissues. The inventors fabricated EHMs and vEHMs and observed them before and after treatment with un-crosslinking solution. The inventors also observed EHMs and vEHMs after five days of culture and before to implantation. FIG. 5A shows P vEHMs are perfused using a custom peristaltic pump system. FIGS. 5B and 5C show tissue compaction and channel diameter over culture period. n=11-14 per group. The inventors observed the histological evaluation of vEHMs next to a patterned vessel and near the tissue edge.

FIG. 6A-6F is a set of graphs showing that implants mature in vivo, and engraftment is improved by in vitro perfusion. The inventors prepared explanted hearts after perfusion with radiopaque dye Microfil®. The inventors also prepared picrosirius red-fast green staining of transverse heart sections showing anterior free wall with infarct and implant. FIG. 6A is a bar graph showing the measurement of infarct size. FIG. 6B is a bar graph showing the anterior wall thickness excluding implants. FIG. 6C is a bar graph showing viable implant size. FIG. 6D is a bar graph showing the histological evaluation of human cell engraftment with Hu-Ku80 staining. FIG. 6E is a bar graph showing the histological evaluation of graft maturation with atrial (MLC-2a) and ventricular (MLC-2v) myosin-heavy chain isoforms. FIG. 6F is a graph showing the measurement of hiPSC-cardiomyocyte sarcomere length in implants. n=3-4 per group.

FIG. 7A-7H shows patterned vasculature increases implant vessel diameter, but in vitro perfusion improves implant vessel organization. The inventors prepared histological images of intra-implant vessels and supportive stromal cells. FIG. 7A is a bar graph shows the distribution of vessel diameters between SMA+ and SMA-vessels. FIG. 7B indicates the percent of SMA-expressing vessels in implants. n=3-4 per group. The inventors produced images of perfused vasculature imaged with OCT in the remote, border, infarct, and implant regions. FIG. 7C shows the tortuosity index. FIG. 7D shows the intra-implant diameter of perfused vasculature imaged with OCT. n=3-4 per group. The inventors produced MicroCT scans of cardiac and intra-implant vasculature overlaid on tissue structure. Measurement of (FIG. 7E) host-implant vascular bridges and intra-implant vessel segment (FIG. 7F) length, (FIG. 7G) diameter, and (FIG. 7H) branching per segment level. n=2-3 per group.

FIG. 8A-8B is a pair of line graphs showing cellular viability and morphology. FIG. 8A shows endothelial cell and FIG. 8B shows hiPSC-cardiomyocyte viability in response to culture in mixed media. n=6-9 per group. The inventors performed histological staining for α-sarcomeric actinin in hiPSC-cardiomyocytes.

FIG. 9A-9H is a set of graphs showing engineered human myocardial tissue compaction and contractile kinetics. FIG. 9A is a line graph showing EHM compaction over one week of mixed media culture. EHM contractile kinetics of (FIG. 9B) upstroke velocity, time to (FIG. 9C) 50% and (FIG. 9D) 90% relaxation (T50 and T90), (FIG. 9E) force frequency response, and (FIG. 9F) maximum capture rate. n=18-21 per group. (FIG. 9G) Prevalence of contractile alternans and (FIG. 9H) relative amplitude of partial-force contractions measured during force-frequency response testing. n=14-19 per group.

FIG. 10 is a bar graph showing wet spun collagen microfiber bundles. Wet spun collagen microfibers are wrapped around themselves four times to create bundled fibers. Collagen microfiber diameter compared to individual microfibers. n=16-18 per group.

FIG. 11A-11D is a set of line graphs showing the echocardiographic metrics of heart function. FIG. 11A shows the measurements of fractional shortening. FIG. 11B shows the measurements of ejection fraction. The left ventricular inner diameter is shown in (FIG. 11C) systole and (FIG. 11D) diastole. n=4-6 per group.

FIG. 12A-12E is a set of bar graphs showing the characterization of human cells in implants. FIG. 12A shows a viable implant size relative to left ventricle size. FIG. 12B shows the density of human cells in implants by Hu-Ku80+ staining. FIG. 12C shows the ratio of Hu-Ku80+ to total nuclei. FIG. 12D shows the measurement of cTnT area in implants. FIG. 12E shows the proliferation assessment of implanted hiPSC-cardiomyocytes by staining for Hu-Ki67 (white arrows) and cTnl. n=3-4 per group.

FIG. 13 is a bar graph showing the implant vascularization and human vascular morphology. The inventors produced images of vessel morphology in implants. FIG. 13 showed measurement of number of vessels across remote, infarct, and implants regions. n=3-4 per group. The inventors observed vascular stains of the remote and infarct regions of sham hearts. The human endothelial cells in NP vEHMs appear as isolated capillary sprouts (white arrows) or as part of larger, host-lined vessels. Human endothelial cells in P vEHMs are only found in chimeric vessels.

FIG. 14A-14D is a set of graphs showing perfusion measurement by OCT and microCT. OCT imaging setup during intralipid perfusion and example scan area with 5× objective. Corresponding preview image after acquisition and final image after contrast-mask averaging of multiple volumes. False-colored image of implant vasculature showed vessel depth. FIG. 14A is a pair of dot plots showing the comparison of vessel diameters as measured by histological analysis. FIG. 14B shows microCT analysis of (FIG. 14C) intra-implant vascular volume and (FIG. 14D) branching per segment level. n=2-3 per group.

FIG. 15A-15B is a pair of graphs showing exploratory bulk RNA sequencing of explanted EHMs. FIG. 15A is a bar graph showing the alignment of sequenced samples to Hg38 (human) and Rn6 (rat) genomes. FIG. 15B shows principal component analysis of human-aligned samples. The inventors performed comparisons of gene regulation across categories of cardiomyocyte and vascular development. n=1-2 per group.

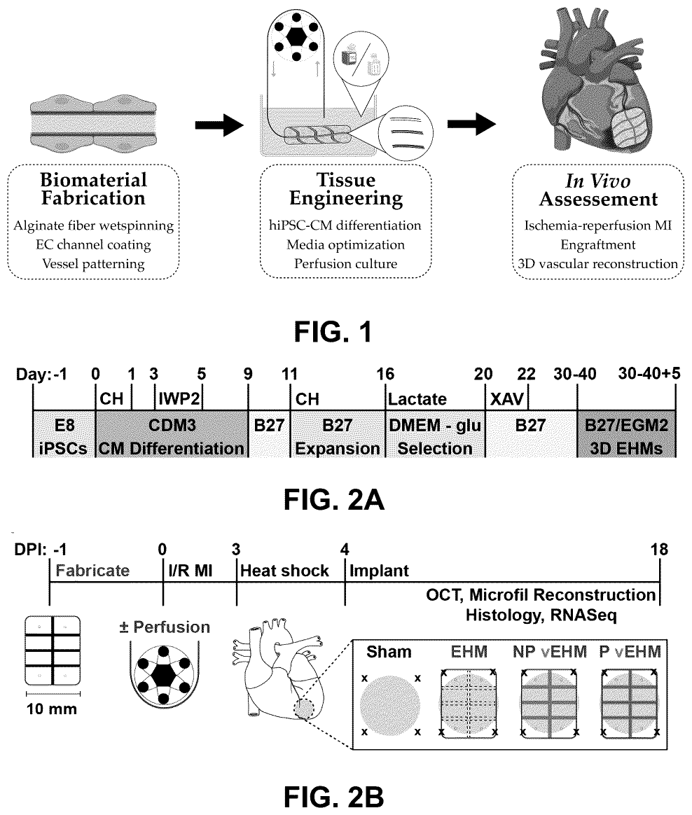

### DETAILED DESCRIPTION OF THE INVENTION

### Industrial Applicability

Vascularization remains a primary concern when developing implantable cardiac tissues for superior engraftment and integration, especially concerning the impact of vasculature on hiPSC-cardiomyocyte survival, development, maturation, and integration in vivo.

The sophistication of cardiac regenerative therapies continues to advance. Several considerations were balanced for design and implementation in this application. hiPSC-cardiomyocytes are immature right after differentiation, as described by Gomez-Garcia, Quesnel, Al-attar, Laskary, & Laflamme, Semin. Cell Dev. Biol. (2021); Guo & Pu, Circ. Res., 126, 1086-1106 (2020); and Dhahri et al., Circulation (2022). Mechanical, electrical, or biomolecular methods can mature the fetal-like contractile apparatus, gene expression, electrophysiology, and metabolism of hiPSC-cardiomyocytes. See Abilez et al., Stem Cells, 36, 265-277 (2018); Querdel et al., Circulation, 143, 1991-2006 (2021); Sun & Nunes, J. Vis. Exp., 2017, 1-8 (2017); Tohyama et al., Cell Stem Cell, 12, 127-137 (2013); Feyen et al., Cell Rep., 32, 107925 (2020); and Miki et al., Nature Commun., 12, 3596 (2021).

Strategies to prevascularize cardiac tissues in vitro provide conduits for rapid perfusion after implantation. See Kant & Coulombe, Acta Biomater., 69, 42-62 (March 2018). Unlike to in situ methods, in which host vasculature grows into and perfuses an implant, in vitro prevascularization allow for substantial control over engineered cardiac tissue design and intentional vascular development, to improve in vivo engraftment of muscle. Munarin et al., Biomaterials, 251, 120033 (2020); Dvir et al., Proc. Natl. Acad. Sci., U.S.A., 106, 14990-14995 (2009); Koffler et al., Proc. Natl. Acad. Sci., U.S.A., 109, 1353-1353 (2012).

Common methods include co-culture of vascular units (e.g., endothelial cells (ECs) or isolated microvessels) to establish homogeneously distributed microvasculature or patterning vessels across larger scales for top-down vascular integration by way of microfabrication techniques such as 3D bioprinting and pattern templating. See Schaefer et al. Tissue Eng. Regen. Med, 12, 546-556 (2018); Sun et al., Sci. Transl. Med, 12, 2992 (2020); Mirabella et al., Nature Biomed. Eng, 1, 0083 (2017); Mirdamadi et al. ACS Biomater. Sci. Eng, 6, 6453-6459 (2020); Kinstlinger et al., Nature Protoc, 16, 3089-3113, (2021); Brady et al., APL Bioeng, 4, 016105 (2020); Redd et al., Nature Commun. 2019, 10, 584; Vollert et al., Tissue Eng. Part A, 20, 854-863 (2014).

The results provided here contribute to the biomedical art in the fields cardiovascular tissue engineering and heart regeneration techniques for both experimental design and analysis of in vitro and in vivo vascular phenomena, contextualized by the needs of cardiac tissue engineering applications. By leveraging existing assays and clinically available technologies for use in different contexts, the innovations are relevant and easily adaptable for benchside, pre-clinical, and translational applications in cardiac tissue engineering.

### Introduction.

Despite ongoing research in hiPSC-CM-based therapeutics for post-myocardial infarction treatment, the inventors noticed a distinct dearth of literature regarding the function of vascularization, particularly at the large-animal and clinical stages. Previous analyses focused instead on contractile and electrophysiological maturation, with little consideration as to the potential benefits of providing vascular conduits on engineered human myocardial tissues development in vivo.

The proper level of maturity for transplantation in vivo that maximizes survival, e.g., in relative ischemia has not been established. The literature suggests that days of in vitro structural maturation can improve engraftment. See Dhahri et al., Circulation (2022); Funakoshi et al., Nature Commun. 12 (2021). The literature also suggests that metabolic maturation harms implanted cell survival. Peters et al., Stem Cells Transl. Med., 11, 1040-1051 (2022).

Cardiomyocytes mature in vivo. An examination of the relationship between cardiomyocyte maturation and engraftment is ongoing. See Funakoshi et al., Nature Commun. 12 (2021); Biagi et al., J. Pers. Med., 11, 374 (2021); and Riegler et al., et al., Circ. Res. 117, 720-730 (2015).

Heterocellular tissue constructs require selection of culture conditions, but optimized growth medium formulations for each of cardiomyocytes, endothelial cells, and other cells imply that a single culture medium is sub-optimal for other cell types. Empirical evaluation of co-culture conditions is sparse in the tissue engineering literature.

The throughput and ease of biomanufacturing is slowed by increasingly complex tissue and vascular geometries. Pedde et al., Adv. Mater., 29, 1-27 (2017); Kant & Coulombe, Acta Biomater. 69, 42-62 (March 2018). The throughput and ease of biomanufacturing is challenged by the difficulties in handling hiPSC-cardiomyocytes and requiring high cardiomyocyte density for syncytium formation. Eschenhagen, Ridders, & Weinberger, J. Mol. Cell. Cardiol., 163, 106-117 (2022); Shadrin et al., Nature Commun., 8, 1825 (2017).

The inventors identified vascularization as a key factor. Robust prevascularization of engineered human myocardial tissues should enable greater engraftment, perfusion, and maturation. The inventors designed a set of cohesive and far-reaching assays to develop a greater understanding of dynamic in vivo vascularization in the context of engineering a cellular therapeutic and its impact on improving engineered human myocardial tissues engraftment and development. Patterning large arteriole-scale vascular channels in an EHM should provide greater avenues for bulk tissue inosculation and perfusion, enabling greater cardiomyocyte engraftment and maturation.

These considerations influenced the design of the vascularized engineered human myocardial tissues (vEHMs), from biomaterial selection and simple vessel patterns to methods of endothelialization, culture, and implantation. No group previously reported on the effects of mixed medium culture on hiPSC-cardiomyocytes or the use of large vessels (400 μm diameter) in dense hiPSC-cardiomyocyte tissue perfused in vitro and implanted in the injured heart.

The invention provides the formation of vEHMs containing patent and perfused arteriole-scale vessels. This formulation allows their robust engraftment onto infarcted myocardium with increased hiPSC-cardiomyocyte engraftment density and improved hierarchical vascularization of vEHMs with prior in vitro perfusion (P vEHM) compared to non-perfused (NP vEHM) or non-vascularized (EHM) controls.

These results show an effective dual remuscularization-revascularization treatment of myocardial infarction that serves as a foundational basis for critical analysis and translational application of vascularization techniques with epicardial delivery of hiPSC-CMs for therapeutic benefit. The contributions of the work to the field support scientific contributions towards the goal of biomanufacturing vascularized hiPSC-CM patches for clinical scale-up and applications.

### Definitions

For convenience, the meaning of some terms and phrases used in the specification, examples, and appended claims, are listed below. Unless stated otherwise or implicit from context, these terms and phrases shall have the meanings below. These definitions aid in describing particular embodiments but are not intended to limit the claimed invention. Unless otherwise defined, the technical and scientific terms have the same meaning as commonly understood by a person having ordinary skill in the biomedical art. A term's meaning provided in this specification shall prevail if any apparent discrepancy arises between the meaning of a definition provided in this specification and the term's use in the biomedical art.

About has the plain meaning of approximately. The term encompasses the measurement errors inherently associated with the relevant testing. When used with percentages, about means ±1%. About or approximately when referring to a value or parameter means to be within a range of normal tolerance in the art, e.g., within two standard deviations of the mean. A description referring to about X describes X.

Biocompatible has the biomedical art-recognized meaning an ability of a material to perform with an appropriate subject response for a specific use. Biocompatible has the further biomedical art-recognized meaning an ability to be in contact with a living system without producing an adverse effect.

Biomaterial has the biomedical art-recognized meaning. A biocompatible matrix can be a biomaterial selected from biopolymers such as a proteins or polysaccharides, for example a biomaterial such as collagen, gelatin, fibrin, a polysaccharide, e.g., hyaluronic acids, chitosan, and derivatives thereof, collagen, chitosan, etc. Vessel structures can be patterned using a sacrificial biomaterial such as alginate, sugars, etc.

Cardiomyocytes (CMs) have the biomedical art-recognized meaning of a cardiac muscle cell. Cardiomyocytes can mature in vivo. Cardiomyocytes can be induced by paracrine signaling from adjacent healthy myocardium. Previous analyses suggested that twenty-forty days of in vitro maturation is beneficial for improving engraftment, though still morphologically immature comparison to true adult cardiomyocytes.

Contractile has the biomedical art-recognized meaning of capable of contracting or causing contraction, such as a muscle fiber.

Degradable has the biomedical art-recognized meaning capable of being decomposed chemically or biologically in a subject's body.

Endothelial cells (EC) have the biomedical art-recognized meaning of the squamous cells forming the lining of blood and lymph vessels and the inner layer of the endocardium.

Engineered cardiac tissue has the biomedical art-recognized meaning of tissue derived by experimental manipulation of pluripotent stem cells, such as human induced pluripotent stem cells (hiPSCs) to differentiate into cardiomyocytes. See Minor & Coulombe, Stem Cells Transl. Med., 1-10 (2022). In vitro prevascularization provides substantial control over engineered cardiac tissue design, with intentional vascular development. In vitro prevascularization can improve in vivo engraftment of muscle.

Engineered human myocardial tissues (EHMs) have the biomedical art-recognized meaning of human tissue derived by experimental manipulation of human pluripotent stem cells, such as human induced pluripotent stem cells (hiPSCs) to differentiate into human cardiomyocytes.

Heart regeneration has the biomedical art-recognized meaning of regeneration of the myocardium after injury to prevent or treat heart failure.

High cardiomyocyte density has the biomedical art recognized meaning. High cardiomyocyte density can be from 10⁶-10⁸ cells/mL. Similar densities have been published in the biomedical art. See Dwyer et al., Bioengineering, 10, 587 (2023); Eschenhagen et al., J. Mol. Cell. Cardiol., 163, 106-117 (2022); Shadrin et al., Nature Commun., 8, 1825 (2017). In a specific example, hiPSCs replated on 24 well plates at a density of 40,000-70,000 cells/cm² and allowed to grow. In another specific example, a casting mix of hiPSC-cardiomyocytes (1.5×10⁷ cells/mL) was prepared.

hiPSC-derived cardiomyocytes (hiPSC-CMs or hiPSC-cardiomyocytes) have the biomedical art-recognized meaning of human-induced pluripotent stem cell-derived cardiomyocytes.

Human-induced pluripotent stem cells or hiPSCs are a type of pluripotent stem cell that can be generated directly from adult cells. Human-induced pluripotent stem cells are a renewable source of human cells.

Inosculation has the biomedical art-recognized meaning of an intercommunication between two or more vessels, such as the cross communication between arteries or veins. Host-driven integration of implanted vascular designs with native vasculature in vivo is called inosculation, or the natural connection and perfusion of disparate vascular beds, which contrasts with direct surgical anastomosis of pre-formed vessels capable of suture retention and withstanding systolic pressure. See Laschke, Vollmer & Menger, Tissue Eng. Part B Rev., 15, 455-465 (2009).

Matrix has the biomedical art-recognized meaning. A biocompatible matrix can be made of a biomaterial selected from biopolymers such as a proteins or polysaccharides, for example a biomaterial such as collagen, gelatin, fibrin, a polysaccharide, e.g., hyaluronic acids, chitosan, and derivatives thereof, collagen, chitosan, etc.

Mid-diameter vessels and mid-scale vessels have the biomedical art-recognized meaning. Mid-diameter vessels are smaller than large arteries and larger than capillaries. Arterioles have the biomedical art-recognized meaning of small bloods vessels that branch off from an artery and carry blood away from the heart to the tissues and organs. Arteriole-scale vessels are known in the biomedical art to be 7 μm to 500 μm in diameter. Meso-diameter arterioles have the biomedical art-recognized meaning. In one example, microCT-based 3D vascular reconstructions showed the presence of mid-diameter vessels and the improved presence of large vessels (>50 μm) in vEHMs perfused in vitro. In another example, large vessels (400 μm diameter) were produced as well as mid-diameter vessels.

Optical Coherence Tomography (OCT) has the biomedical art-recognized meaning. Optical coherence tomography (OCT) and optical coherence tomography angiography (OCTA) are non-invasive imaging tests based on low coherence interferometry to resolve vascular geometry using light refracted from particulate solutions flowing through blood vessels. See Redd et al., Nat Comm., 10(1), 1-14 (2019). The Murry group showed the use optical coherence tomography (OCT) angiography to image cardiac vasculature and vascularized implants as proof of concept. This analysis was not performed in tissues containing human cardiomyocytes.

Packaging has the plain meaning of material used to enclose or contain something, such as a covering wrapper or container. See Merriam Webster Dictionary online. Packaging can be used for the coordinated system of preparing goods for transport, warehousing, logistics, sale, and end use.

Patterned vessels have the biomedical art-recognized meaning.

Subject, Patient, or Host has the biomedical art-recognized meaning of an animal, e.g., a human, to whom a treatment can be administered.

Tissue engineering is the use of a combination of cells, engineering, and materials methods, and suitable biochemical and physicochemical factors to improve, mimic, or replace biological tissues. Tissue engineering involves using a tissue scaffold to form new viable tissue for a biological or medical purpose. The phrase tissue engineering is often interchangeably used with regenerative medicine.

Vascularization has the biomedical art-recognized meaning of the process of growing blood vessels into a tissue to improve oxygen and nutrient supply.

Vascularized engineered human myocardial tissues (vEHMs) have the biomedical art-recognized meaning.

Vascularized engineered human myocardial tissues with no perfusion (NP vEHM) have the biomedical art-recognized meaning.

Vascularized engineered human myocardial tissues with prior in vitro perfusion (P vEHM) have the biomedical art-recognized meaning.

Unless otherwise defined, scientific and technical terms used with this application shall have the meanings commonly understood by persons having ordinary skill in the biomedical art. This invention is not limited to the method, protocols, reagents, etc., described herein and can vary.

This specification does not concern a process for cloning humans, methods for modifying the germ line genetic identity of humans, uses of human embryos for industrial or commercial purposes, or procedures for modifying the genetic identity of animals likely to cause them suffering with no substantial medical benefit to humans or animals resulting from such processes.

Guidance from Materials and Methods

Persons having ordinary skill in the biomedical art can use these materials and methods as guidance to predictable results when making and using the invention:

Methods of inducing myocardial infarction can vary, leading to different injury and remodeling responses. Permanent ligation of the left anterior descending artery is one of the most common techniques as it creates a massive acute transmural injury. Martin et al., Preclinical models of myocardial infarction: from mechanism to translation. British Journal of Pharmacology, 1-22 (August 2021). This injury often results is higher mortality, as opposed to ischemia-reperfusion (IR) methods with enable restoration of blood flow after an allotted time of ischemia. Ischemia-reperfusion myocardial infarction is a clinically relevant model of ischemic preconditioning and reperfusion injury because it parallels the revascularization treatments that are the standard of clinical care for myocardial infarction. Martin et al., Preclinical models of myocardial infarction: from mechanism to translation. British Journal of Pharmacology, 1-22 (August 2021). This model achieves similar transmural infarctions equivalent to that of permanent ligation methods, though this depends on the duration of ligation.

Ablative cryoinfarction models are another model of acute myocardial infarction, which use liquid nitrogen-frozen probes to create cryoinjuries that mimic myocardial infarction, though with less remodeling and scarring. While less clinically relevant, this method creates more consistent injuries across animals. See, Martin et al., Preclinical models of myocardial infarction: from mechanism to translation. British Journal of Pharmacology, 1-22 (August 2021); Lindsey et al. Guidelines for experimental models of myocardial ischemia and infarction. American Journal of Physiology-Heart and Circulatory Physiology 314, H812-H838 (2018). Chronic models use ameroid/angioplastic constriction, delayed implantation by three-four weeks, or hyperlipidemic/atherosclerosis-inducing diets to model chronic heart failure. These models may be the most clinically-similar to patient incidence of heart failure as it models damage due to relatively slow constriction of blood flow over a longer period of time. Limited analyses in hiPSC-cardiomyocytes implantation in chronic myocardial infarction models have suggested subpar engraftment and lack of functional improvement. Eschenhagen, Ridders, & Weinberger, How to repair a broken heart with pluripotent stem cell-derived cardiomyocytes. Journal of Molecular and Cellular Cardiology 163, 106-117 (February 2022); Fernandes et al., Human embryonic stem cell-derived cardiomyocytes engraft but do not change cardiac remodeling after chronic infarction in rats. Journal of Molecular and Cellular Cardiology 49, 941-949 (December 2010).

Evaluating the effectiveness of vascularization techniques historically relied on the gold standard of static 2D histological analysis, and newer methodologies show promise in providing greater insight into vascular remodeling in the infarcted heart. See Kant & Coulombe, Acta Biomater. 69, 42-62 (2018). Techniques include microcomputed tomography (microCT)-assisted reconstructions, optical coherence tomography (OCT) imaging, and fluorescence imaging enable vascular visualization down to the capillary scale and mapping of perfused vessels. See Weyers et al., J. Am. Heart Assoc. 2 (2013) 1-13; Weyers et al., PLoS One, 15 (2020) e022778; Brady et al., APL Bioeng., 4 (2020) 016105; Redd et al., Nature Commun. 10 (2019) 1-14; Qin et al., Phys. Med. Biol., 61, 7536-7550 (2016); Vinegoni, Aguirre, Lee, & Weissleder, Nature Protocols, 10, 1802-1819 (2015); Merz, et al., Nature Commun., 10, 1-14 (2019), and Anbazhakan et al., Nature Cardiovasc. Res. 1 775-790.

hiPSC-cardiomyocyte differentiation, expansion, and lactate selection. Directed differentiation of hiPSCs into cardiomyocytes was achieved via multi-stage biphasic modulation of the Wnt signaling pathway in defined conditions. See FIG. 2A. See also Lian et al., Nature Protoc., 8, 162-175 (2012); Burridge et al., Nature Methods, 11, 855-860 (2014); Rupert, Irofuala, & Coulombe, PLoS One, 15, e0230001 (2020); and Buikema et al., Cell Stem Cell, 27, 50-63.e5 (2020). hiPSCs were replated on Matrigel®-coated (Corning) 24 well plates in E8 with 10 μM Y-27632 (Rock Inhibitor, RI, USA; Tocris) at a density of 40,000-70,000 cells/cm². The next day (DO), hiPSCs were treated with 4-5 μM CHIR 99021 (Chiron; Tocris) in CDM3 medium for twenty-four hours. See Burridge et al., Nature Methods, 11, 855-860 (2014). On day 3 (seventy-two hours after Chiron treatment) cells were treated with 5 μM IWP2 (Tocris) for two days, then fed CDM3 every other day until beating began (day 8-12). On day 9, hiPSC-cardiomyocytes were fed RPMI 1640 with B27 supplement (RPMI B27; Gibco) for two days, then on day 11 harvested with TrypLE Select 10× (Gibco) and re-plated on Matrigel®-coated 15 cm² plates at 25,000 cells/cm² in RPMI B27 containing 2 μM Chiron and 10 μM RI to encourage cardiomyocyte expansion. Buikema et al., Cell Stem Cell, 27, 50-63.e5 (2020). hiPSC-cardiomyocytes were fed fresh RPMI B27 with 2 μM Chiron every two days until 90% confluent (4-6 days). Cells were then washed with DPBS and fed DMEM-glucose (Gibco) containing 4 mM sodium lactate (MilliporeSigma) every other day for four days to eliminate non-cardiomyocytes from culture as described by Tohyama et al., Cell Stem Cell, 12, 127-137 (2013). This was followed by two days of treatment with 4 μM XAV939 (Tocris) in RPMI B27 to encourage cell cycle exit of hiPSC-cardiomyocytes. See Funakoshi et al., Nature Commun. 12 (2021). Expanded, purified hiPSC-cardiomyocytes were then maintained in RPMI B27 containing 100 μg/mL penicillin-streptomycin (pen-strep; MilliporeSigma) and used between days 30-40 of differentiation for downstream assays. The average purity of hiPSC-cardiomyocytes used for in vitro and in vivo assays was 94.0% and 85.05%, respectively, by flow cytometry staining for cardiac troponin T (cTnT).

hiPSC maintenance. The WTC-11 GCaMP6f hiPSC line was used in assays (Dr. Bruce Conklin, The Gladstone Institutes). hiPSCs were maintained on 10 cm² dishes coated with 5 μg/mL vitronectin (Thermo Fisher) in a cell culture incubator (37° C., 5% CO 2). hiPSCs were maintained in Essential 8 (E8) medium (Gibco) and passaged every four-five days at 80% confluency with versene (0.5 M EDTA (Fisher) and 1.1 mM D-glucose (MilliporeSigma) in DPBS without calcium and magnesium (Gibco)) onto new vitronectin-coated plates.

Cardiac fibroblast maintenance. Primary normal adult ventricular human cardiac fibroblasts (hCFs; MilliporeSigma) were maintained on 15 cm² culture dishes in hCF medium consisting of DMEM\F-12, 10% fetal bovine serum (FBS; Gibco), 4 ng/mL bFGF (Stemgent), and 100 μg/mL pen-strep. Human cardiac fibroblasts were passaged every four-five days at 80% confluency using 0.05% trypsin (Gibco) in versene and frozen back in hCF medium with 10% dimethylsiloxane (DMSO; Fisher). The assays used human cardiac fibroblasts directly from thaw between passages 3-6. Other sources of cardiac fibroblasts or stromal cells may be used, including hiPSCs, adipose tissue, bone marrow, etc.

Endothelial cell maintenance. Human umbilical vein endothelial cells (ECs; Lonza) were maintained on 0.1% gelatin-coated (MilliporeSigma) 15 cm² culture dishes in EGM-2 Medium (Lonza). Endothelial cells were passaged every four-five days at 80% confluency using 0.05% trypsin in versene, and frozen back in EGM-2 with 10% DMSO. The assays used endothelial cells between passages 3-7.

Cellular viability, proliferation, and morphological assays. Endothelial cell and hiPSC-cardiomyocyte viability was assessed by plating cells on 0.1% gelatin-coated or MG-coated 6 well plates, respectively, and feeding with either control medium (EGM-2 for endothelial cells; 0/100, or RPMI B27 for hiPSC-cardiomyocytes; 100/0), or mixed medium consisting of either 70% RPMI B27 and 30% EGM-2 (70/30) or 50% RPMI B27 and 50% EGM-2 (50/50). 100 μg/mL pen-strep was added to the media. Daily images were taken to assess the number of live and dead cells in five separate microscope fields per condition. The morphology and structure of hiPSC-cardiomyocytes was assessed with immunohistochemical staining.

Mechanical characterization of engineered human myocardial tissues. Culture molds were fabricated using a replica molding technique reported by Munarin, Kaiser, Kim, Choi, & Coulombe, Tissue Eng. Part C. Methods. 23, 311-321 (2017) and Kaiser, Munarin, & Coulombe, J. Vis. Exp., 2018, 1-7 (2018). Vector-based designs were created in Adobe Illustrator (Adobe Inc., San Jose, CA, USA) with a 9×3 mm culture area, then laser-cut into a ¼-inch acrylic sheet using a Universal Laser 8 Systems 6.75 Laser Cutter (ULS) at the Brown Design Workshop (Brown University). Sylgard 184 (Dow) was cast into the acrylic templates and cured overnight at 60° C. The cured CAN molds were then removed from the acrylic negative and sterilized in an autoclave at 121° C. for thirty minutes. hiPSC-cardiomyocytes were harvested between days of differentiation. hiPSC-cardiomyocytes were resuspended at a density of 1.5×10⁷ cells/mL and mixed with 5% human cardiac fibroblasts from thaw by number and rat tail-derived collagen I (final concentration 1 mg/mL; Advanced BioMatrix). The suspension was cast into each trough of the PDMS mold and allowed to set for thirty minutes at 37° C. before feeding with either 100/0, 70/30, or 50/50 medium. Tissues were cultured for one week and paced at 1 Hz using a C-Pace EM Stimulator (IonOptix, Westwood, MA, USA). Mechanical testing was performed as previously described by Rupert, Kim, Choi, & Coulombe, Stem Cells Int. 2020 (2020). Engineered human myocardial tissues were mounted between a 5 mN load cell (Aurora Scientific) and a high-speed length controller in a temperature-controlled bath of Tyrode's solution. Initial length (L₀) was set at just above slack length. Tissues were stretched in 5% length step increments to 130% of L₀ and resulting force generation at each step was measured under a 1 Hz field stimulus. Tissues were then electrically paced from 1 Hz to 4 Hz in 0.5 Hz increments to determine maximum capture rate before returning length to L₀. Tissues were then immediately fixed for downstream immunohistochemistry. Passive stiffness and active force kinetics were measured using a custom MATLAB code (Mathworks Inc.).

Gelatin-alginate fiber wet spinning. Wet spinning of sacrificial fibers was adapted from Kant, Bare, & Coulombe, Tissue Eng. Part A, 27 (19-20), 1290-1304 (October 2021). A 1% w/v sodium alginate (MilliporeSigma) solution was prepared in deionized (DI) water. The solution was loaded into a 5 mL syringe fitted with a 30 G hypodermic needle. Crosslinking solution was made by dissolving 100 mM calcium chloride (Acros Organics) and 2.5% w/v gelatin (MilliporeSigma) in DI water. Alginate was extruded into the crosslinking bath via a syringe pump at a rate of 50 μL/sec. The resulting cross-linked gelatin-alginate (GA) alginate fibers were collected on a mandrel. Wet spun fibers were sprayed briefly with 70% ethanol to disinfect and stored in a biosafety cabinet. Gelatin-alginate fiber diameter was measured using ImageJ. Node measurements were determined by counting the number of nodes that seemed normalized over the length of the coated fiber.

Collagen microfiber wet spinning was adapted as previously described by Kaiser et al., Tissue Eng. Part C Methods, 25, 687-700 (2019), to create bundled collagen microfibers. Thirteen mg/mL collagen I isolated from rat tails was extruded using a syringe pump through a spinneret and ensheathed in a coaxial flow of a high-viscosity polyethylene glycol-based neutralization buffer to form collagen microfibers. The formed microfibers were then washed in a bath of 70% ethanol and collected on a mandrel to dry. Microfiber bundles were formed by wrapping collagen microfibers around the same location on the mandrel four times and briefly rewetting the fibers with 70% ethanol to form robust bundles. Microfiber bundle diameter was measured in ImageJ.

Engineered human myocardial tissue fabrication for in vivo assays. Polydimethylsiloxane (PDMS) molds were fabricated to create 10×12 mm engineered human myocardial tissues for implantation assays and prepared as described above. In the biosafety cabinet, gelatin-alginate fibers and collagen microfibers were aseptically embedded in autoclaved PDMS molds as described by Kant, Bare, & Coulombe, Tissue Eng. Part A, 27 (19-20), 1290-1304 (October 2021), using a 25 G needle to form an array pattern of gelatin-alginate fibers through the center of the tissue with a set of collagen microfibers along the longitudinal edges of the mold. The molds were then adhered to the bottom of untreated 6 well plates with silicone adhesive (Dow). A casting mix of hiPSC-cardiomyocytes (1.5×10⁷ cells/mL), 5% human cardiac fibroblasts and collagen I was prepared as described with volumes scaled to accommodate the larger culture molds and kept on ice. Endothelial cells were thawed, resuspended at a density of 2.5×10⁷ cells/mL in a coating solution of EGM-2, 1% w/v gelatin, 0.25% w/v alginate, and 1 mg/mL collagen I, and carefully pipetted onto the gelatin-alginate fibers to coat the surface. The casting mix of hiPSC-cardiomyocytes, human cardiac fibroblasts, and collagen I was then immediately seeded into the PDMS mold around the fibers and allowed to gel for thirty minutes at 37° C. to form vascularized engineered human myocardial tissues. vEHMs were maintained in 50/50 medium and paced at 1 Hz under field stimulation. Control engineered human myocardial tissues were fabricated following the same procedure except the coating solution was devoid of endothelial cells.

Gelatin-alginate fiber un-crosslinking and perfusion culture. After tissue formation overnight, gelatin-alginate fibers embedded in engineered human myocardial tissues were uncrosslinked by a three hour incubation in un-crosslinking medium, consisting of calcium-free DMEM (Gibco), 2 mM GlutaMAX (Gibco), 1.5 mM sodium citrate (Fisher), and 100 μg/mL pen-strep. Uncrosslinking medium was then manually perfused through the channels to wash out any remaining gelatin-alginate and confirm patency, followed by a medium change back into EHM medium. Dynamic perfusion culture of patterned vessels in vascularized engineered human myocardial tissues was achieved with a custom-built Arduino-controlled peristaltic pump, enabling independent recirculating perfusion of up to three tissues simultaneously in vitro. A diagram of an arduino-based peristaltic pump designed for use as a perfusion bioreactor for dynamic perfusion culture of engineered cardiac tissues for regenerative medicine is available at Kant, “Peristaltic Pump and Perfusion Bioreactor for Cardiac Tissue Engineering”, Harvard Dataverse, V1 (2022). https://doi.org/10.7910/DVN/KARO3F. The attached files provide the .stl schematics for 3D-printing the peristaltic pump pieces and accessory items, illustrator design for the laser-cut acrylic housing, wiring diagram, and bill of materials.

The media were directed through a reservoir before infusion to prevent embolism by bubbles and dampen the flow profile to an average of 7.28 mL/hr. The media were partially exchanged (about half of total recirculating volume) every other day by replacing medium buildup in the well of the plate. Control and non-perfused tissues were not connected to perfusion devices and fed every other day with EHM medium. vEHM compaction and patterned vessel diameter were evaluated using ImageJ. Perfusion device design files are free to access and download at the Harvard Dataverse.

Implantation of engineered human myocardial tissues on infarcted hearts and explant. The animal procedures used approved ethical guidelines. Nude, athymic rats (eight weeks, male, 200 g) underwent ischemia-reperfusion myocardial infarction using protocols reported by Munarin, Kant, Rupert, Khoo, & Coulombe, Biomaterials, 251, 120033 (2020) and Munarin, Kaiser, Kim, Choi, & Coulombe, Tissue Eng. Part C. Methods. 23, 311-321 (2017). Rats were anesthetized with 2-3% isoflurane. Rats were then induced with 100 mg/kg ketamine and 90 mg/kg xylazine and placed on a ventilator. A thoracotomy was performed to expose the heart and the left anterior descending artery was ligated with 4-0 polypropylene suture for sixty minutes to induce ischemic myocardial infarction, followed by reperfusion by removal of the suture and closure of the chest. Three days after myocardial infarction, engineered human myocardial tissues were prepared for implantation by performing a heat shock protocol described by Laflamme et al., Nature Biotechnol., 25, 1015-1024 (2007), consisting of incubation at 42° C. for one hour. Animals were additionally given daily subcutaneous injections of 30 mg/kg cyclosporine-A to mitigate mitochondrial permeability transition pore opening starting the day before implantation. The next morning, four days after myocardial infarction, engineered human myocardial tissues were treated with 100 ng/mL IGF-1 (RD Systems) and 200 nM cyclosporine-A (MilliporeSigma) one hour before the implant procedure. Animals underwent a second thoracotomy to implant engineered human myocardial tissues over the infarcted region of the left ventricle. Sham animals received sutures but no implants.

Echocardiography measurements were performed under mild sedation using a Vivid 7 Dimension Ultrasound System (GE) with a pediatric 10 S 4-10 MHz transducer. Longitudinal and short-axis views of the left ventricle at the mid-papillary level were taken to assess cardiac function before myocardial infarction (baseline), four days post-injury (DPI; before implantation), and before sacrifice at 18 DPI (two weeks after implantation). Echocardiography data was additionally used as exclusion criteria (fractional shortening (FS)<45%) to ensure consistent myocardial infarctions with reduced FS. At fourteen days, hearts were prepared for vascular morphological and reconstructive analysis by the method of Munarin, Kant, Rupert, Khoo, & Coulombe, Biomaterials, 251, 120033 (2020) and Weyers et al., J. Am. Heart Assoc., 2, 1-13 (2013). Rats were injected with 750 USP/mL heparin (Meitheal Pharmaceuticals) via tail vein injection, then underwent a thoracotomy to expose the heart and aorta. The superior vena cava and innominate, left common carotid, and left subclavian branches of the aortic arch were ligated, then the abdominal aorta was cannulated with an 18 G catheter to provide anterograde perfusion access to the cardiac microcirculation via the aortic root. The cannula was connected to a perfusion system and the inferior vena cava cut to provide an outlet. Perfusion buffer containing 1 g/L adenosine (MilliporeSigma) in DPBS was perfused for three minutes to clear blood then briefly perfused with 150 mM potassium chloride (Fisher) to arrest the heart in diastole. After another two minutes of perfusion buffer, 4% paraformaldehyde (PF; MilliporeSigma) was perfused for ten minutes to fix the vasculature, followed by explant of the heart, lungs, and aortic arch which were fixed overnight in 4% PF.

Optical coherence tomography angiography. To prepare for ex vivo microangiography, hearts were re-cannulated at the aortic arch using a male luer adapter before the innominate branch, connected to a perfusion system, and perfused with 20% Intralipid (Fresenius Kabi) solution at a pressure of 50-60 mmHg for spectral domain optical coherence tomography imaging (OCT; Thorlabs). OCT acquisition used a large-bandwidth near-infrared (NIR) light source with a 2048-pixel line-scan camera to achieve 147,000 A-scans/s focused on areas about 100-200 μm below the surface of the heart or implant. A 5× objective (3×3 mm FOV) or 10× objective (1.5×1.5 mm FOV) was used for image acquisition. For each acquisition phase, two-four B-scans were taken at each y position with twenty volumetric scans each. A custom contrast-mask averaging process was used to process raw imaging data and remove visual artifacts due to movement. Tortuosity measurements were performed by measuring the length of a vessel starting and terminating at branch points and normalizing by to the shortest distance between the start and end. Diameter measurements from microangiograms were measured manually in ImageJ.

Vascular reconstructive analysis and immunohistochemistry. After OCT microangiography, hearts were cleared of Intralipid then manually perfused with Microfil® (Flow Tech Inc.), allowed to cure, and scanned with a microCT imaging system (Quantum GX2) at a voxel size of 50 μm. Raw image data was processed using custom MATLAB code and segmented in SimVascular to isolate implant vasculature. See Updegrove et al., Ann. Biomed. Eng., 45, 525-541 (2017). Host-implant bridging was manually determined from SimVascular segmentations. Vasculature was then post-processed using a connectivity algorithm to restore broken vessels due to air bubbles during Microfil® perfusion and binarization artifacts. Jiang et al., J. Invest. Dermatol., pp. 528-536 (2011), The resulting vasculature was reconstructed for visualization and quantitative analysis, including vessel segment length, diameter, branching, and volume. After scanning, hearts were processed into 2 mm slices, processed into paraffin or frozen blocks, and sectioned into 14 μm slices for immunohistochemical analysis. Infarct size was visualized and measured using picrosirius red-fast green stains of heart sections at mid-papillary level. Sections were treated with primary antibodies overnight at 4° C. followed by secondary antibodies and nuclear counterstain for one hour at room temperature. Cover slips were mounted using ProLong AntiFade Glass Mountant (Invitrogen). Histological stains were imaged using an Olympus FV3000 Confocal Microscope or Olympus FV200 Slide Scanner (Olympus; Leduc Biolmaging Facility, Brown University), and processed in ImageJ. Primary and secondary antibodies used are provided in TABLE 1. Human cell density and viable implant size were measured in ImageJ from histological stains of cTnT and human-specific Ku80 (Hu-Ku80) and normalized by measured area of human grafts. Engraftment was determined by normalizing density measurements to implant size. Myosin light chain 2 (MLC2) measurements were determined in ImageJ by thresholding histological stains of atrial (MLC2a) and ventricular (MLC2v) isoforms and measuring area. Sarcomere length was determined in histological stains of cTnT by measuring the length of between 3-10 sarcomeres in a straight line and diving by the number of encompassed sarcomeres. Vessel density and diameter were measured in histological stains of isolectin B4 and Hu-Ku80 in the remote (healthy), infarct, and implant regions of each heart, and normalized by imaged area. Smooth muscle actin (SMA) measurement was performed in viable implant areas by cross-registering sections with adjacent Hu-Ku80 stains and normalized to total vasculature.

Exploratory bulk RNA sequencing. A subset of implants were explanted at the 14 day timepoint for immediate RNA isolation using Tri Reagent solution (Invitrogen) followed by on-column processing and washing using a Reliaprep RNA Miniprep System (Promega). Yield and purity were analyzed on a Nanodrop ND-1000 UV-Vis Spectrophotometer (Thermo Fisher) before shipment for next generation sequencing (Azenta Life Sciences). The RIN score of the samples was verified to be above 7.5 before continuing with library preparation and RNA sequencing (RNAseq).

The open-source web-based platform, Galaxy, was used to perform standard quality control analysis, trimming, alignment of the RNAseq raw data to appropriate reference genomes, and generation of count matrices. Afgan et al., Nucleic Acids Res., 46, W537-W544 (2018). Sequence quality was assessed through FASTQC and trimming of the Illumine adaptor sequences was performed with Trimmomatic. See Bolger, Lohse, & Usadel, Bioinformatics. 30, 2114-2120 (2014). Sequences were mapped to the human (hg38) and rat (rn6) genome using HISAT2, with alignment scores calculated from percentage of aligned reads. Count matrices of gene counts per samples were generated using featureCounts, which were then used to generate graphs of relative gene expression in individual samples.

To explore the dataset and assess DE gene expression and gene ontology (GO) enrichment, the inventors generated a custom Bioconductor-based R script in which initial gene filtering was achieved using the function koverA in the genefilter package. Filtered genes were used as the input to the package DESeq2 to perform size-based normalization and DGE analysis on the data set. In this dataset, the inventors assumed equal variance across groups to account for conditions with a sample size of 1. Principal components analysis (PCA), unsupervised clustering, dispersion modeling and heat mapping of gene expression differences among samples were performed with the complete filtered gene list and the differentially expressed genes (DEGs).

Gene ontology (GO) analysis was performed on sets of upregulated or downregulated genes. A false discovery rate (FDR) of <0.05 was statistically significant for both DGE and GO analysis.

Data and statistical analysis. RNAseq DEG data were analyzed and graphed in RStudio. All other data were analyzed and graphed in Prism 9 (GraphPad). Statistics were calculated using one-way or two-way ANOVA with Tukey-Kramer post-hoc analysis. p-values <0.05 were statistically significant. Data are expressed as the mean±standard error.

TABLE 1
Antibodies used in immunohistochemical staining.

| Antibody/Fluorophore | Dilution | Catalog No.; Vendor |
|---|---|---|
| Mouse monoclonal anti-α | 1:500 | A7811-.2ML; MilliporeSigma |
| sarcomeric actinin | | |
| Mouse monoclonal anti-cTnT | 1:100 | MA5-12960 UL; Invitrogen |
| Rabbit monoclonal anti- | 1:150 | ab150301; Abcam |
| smooth muscle actin | | |
| Mouse monoclonal anti- | 1:150 | PIMA511547; Invitrogen |
| smooth muscle actin | | |
| Mouse monoclonal anti-human CD31 | 1:100 | M082301-2; Agilent |
| Rabbit polyclonal anti-MLC2v | 1:100 | 10906-1-AP; ProteinTech |
| Mouse monoclonal anti-MLC2a | 1:100 | 311 011; Synaptic Systems |
| Rabbit polyclonal anti-cTnl | 1:150 | ab47003; Abcam |
| Rabbit polyclonal anti-Connexin-43 | 1:200 | C6219-100UL; MilliporeSigma |
| Rabbit monoclonal anti-human Ku80 | 1:150 | 2180S; Cell Signaling |
| | | Technologies |
| Bisbenzimide H 33342 | 1.5 | B2261-100MG; MilliporeSigma |
| trihydrochloride (Hoechst) | μg/mL | |
| Goat anti-mouse/rabbit Alexa Fluor 488 | 1:300 | A-1100; A-11008; Invitrogen |
| Goat anti-rabbit/mouse Alexa Fluor 594 | 1:300 | A11012; A-11005; Invitrogen |
| Griffonia simplicifolia isolectin B4 | 1:200 | DL-1207-.5; Vector Labs |
| Dylight 594-labeled | | |
| Mouse anti-human Ki67 FITC-labeled | 1:50 | 612472; BD Biosciences |

Other methods. The inventors developed a suite of unbiased and open-source analysis tools to evaluate vascular migration from aortic rings using histological stains or micrographs. More broadly, these tools are widely applicable for other tissue engineering analyses, such as the use of the nearest neighbors algorithm to analyze density or infiltration into a tissue. Together, these innovations provide new options for tissue engineers to evaluate host-implant vascular migration in a controlled but complex in vitro environment to analyze vascular efficacy of a tissue engineered therapeutic before in vivo implementation.

The following EXAMPLES are provided to illustrate the invention and should not be considered to limit its scope.

### Example 1

### Patterned Arteriole-Scale Vessels Enhance Engraftment, Perfusion, and Vessel Branching Hierarchy of Engineered Human Myocardium for Heart Regeneration

The inventors developed a flexible in vitro system for fabrication and dynamic perfusion culture of vascularized engineered human myocardial tissues. The inventors showed successful inosculation and hierarchical vascular bed development in a remuscularization approach to heart regeneration in a rat model of ischemia/reperfusion myocardial infarction.

Implantation of vascularized engineered human myocardial tissues showed that endothelialized channels contributed to increased vessel diameter and branching within implants. In vitro perfusion of vEHMs before implant provided the greatest improvements in hiPSC-cardiomyocyte engraftment, organization of vasculature for efficient hierarchical perfusion by increasing diameters and reducing tortuosity, recruitment of SMA+ vascular support cells, and potentially also hiPSC-cardiomyocyte development.

This EXAMPLE increases the understanding of the design criteria for fabricating patterned vascularized engineered human myocardial tissues and their direct benefits on engineered tissue survival in vivo in a co-vascularization-remuscularization strategy for therapeutic regeneration of the heart after myocardial infarction.

Medium composition influences hiPSC-cardiomyocyte and endothelial cell structure and function. A primary consideration of fabricating complex, multi-cellular engineered human myocardial tissues is the impact of culture medium on each cell population due to varying levels of ions, growth factors, and other supplements. Endothelial cells were maintained in EGM-2. hiPSC-cardiomyocytes were cultured in RPMI B27. The inventors evaluated the impacts of mixed medium culture on each cell type individually to determine the most acceptable compromise. The inventors examined endothelial cell and hiPSC-cardiomyocyte viability in 2D culture over five days in their standard medium (100/0 or 0/100 CM/EC medium), 70/30 CM/EC mixed medium, or medium. See FIG. 8. Despite no significant differences in hiPSC-cardiomyocyte viability, hiPSC-cardiomyocytes in mixed medium conditions showed markedly increased sarcomere development and hypertrophy relative to hiPSC-cardiomyocytes in control cardiomyocyte medium, due in part to the low presence of factor-rich FBS in EGM-2 to promote protein production.

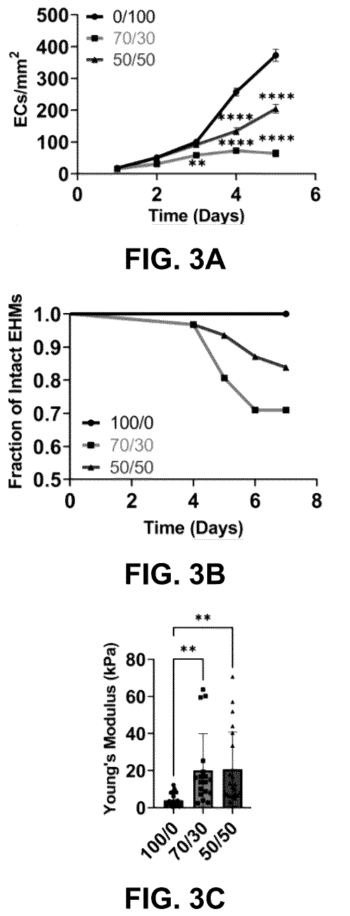

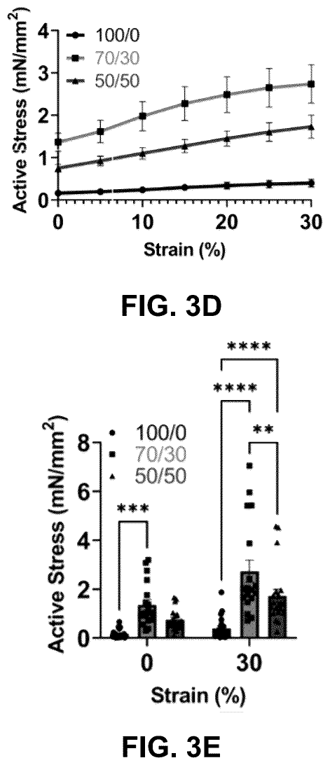

When culturing endothelial cells in mixed media conditions, there was a significant decrease in day 1 attachment of endothelial cells in 70/30 medium relative to control EGM-2, suggesting that medium composition functions in endothelial cell binding interactions. Endothelial cells in control medium showed a characteristic cobblestone morphology and rapidly proliferated until confluence at five days. Endothelial cells cultured in mixed medium proliferated markedly slower, resulting in significantly fewer cells present in the wells of the 70/30 condition as early as day 3 (100/0: 100.46±7.39 cells/mm²; 70/30: 57.93±5.53 cells/mm², p=0.0036), and showing in the 50/50 condition by day 4 relative to control (100/0: 257.36±12.59 cells/mm²; 50/50: 134.37±11.19 cells/mm², p<0.0001). Although endothelial cells in either composition did not reach confluency within five days, endothelial cells in 70/30 medium had the slowest proliferation rate and had qualitatively irregular, spread morphologies. See FIG. 3A. Endothelial cells in 50/50 medium exhibited primarily a cobblestone morphology in more dense areas, with few cells adopting a spread morphology.

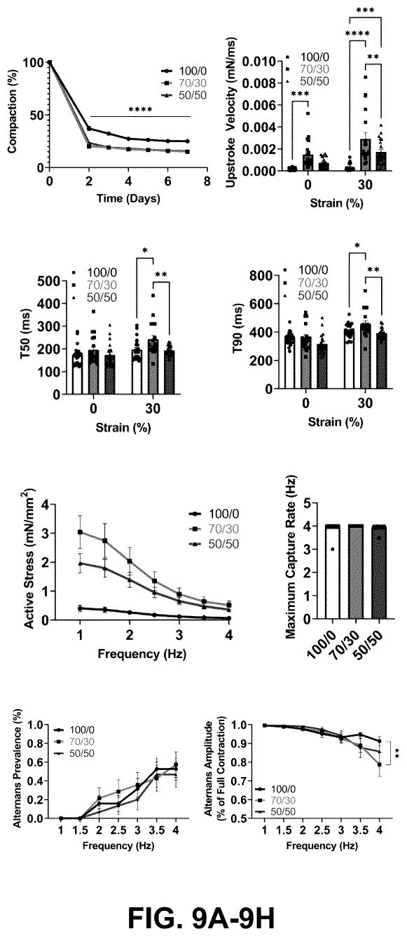

To evaluate the functional consequences of mixed medium compositions on hiPSC-cardiomyocytes in 3D culture, the inventors fabricated small (9×3 mm) linear engineered human myocardial tissues. The inventors observed increased compaction of engineered human myocardial tissues in 70/30 medium relative to 50/50 medium (see FIG. 9A), and particularly many instances of hypercompaction, necking, and failure of the EHMs (p=0.0079 by Mantel-Cox test). See FIG. 3B. After seven days of in vitro culture, the inventors mechanically analyzed the tissues followed immediately by fixation and histological analysis. Mechanical evaluation was performed only on intact tissues. This evaluation could underestimate the true mechanics of groups that experienced greater compaction and necking. Tissues exhibited significantly greater stiffness in mixed media culture (70/30: 20.18±4.67 kPa, p=0.0071; 50/50: 20.10±4.39 kPa, p=0.0033) relative to control tissues (4.08±0.71 kPa). See FIG. 3C. Active twitch stress was also significantly different across groups (see FIG. 3D-3E), with 70/30 tissues generating greater twitch stress (1.37±0.21 mN/mm², p=0.0006) relative to control tissues (0.16±0.04 mN/mm²) at 0% stretch, and both mixed media groups (70/30: 2.74±0.46 mN/mm², p<0.0001; 50/50: 1.73±0.27 mN/mm², p<0.0001) outperforming control tissues (0.40±0.1 mN/mm²) at 30% stretch, in line with morphological observations of increased myofilament development and hypertrophy. The inventors also observed similar significant increases in upstroke velocity, T50, and T90. See FIGS. 9B-9D).

The inventors performed force-frequency response analysis. The inventors noted that despite condition, the tissues showed an inverse relationship between pacing frequency and force generation (FIG. 9E) and could follow pacing up to 4 Hz (FIG. 9F). During the force-frequency response protocol, tissues began exhibiting contractile alternans at 2 Hz, which increased in prevalence with pacing frequency despite condition (FIG. 9G). Calculating relative amplitude compared to full contraction trended towards more severe partial-force contractions in mixed media conditions, which reached significance at 4 Hz between 70/30 (78.7±6.3%, p=0.0035) and control (91.2±2.5%). See FIG. 9H. See Rupert, Kim, Choi, & Coulombe, Stem Cells Int. 2020 (2020).

Immunohistochemical staining of tissues following mechanics analysis showed noticeable improvements in sarcomere development and mild hypertrophy, corroborating 2D histological data, with greatest sarcomere density in the 70/30 medium group.

These results indicate that a mixed medium composition of EGM-2 and RPMI B27 improves hiPSC-cardiomyocytes contractility likely through increased sarcomere development, but negatively affects Endothelial cell viability and proliferation. The 70/30 improves engineered human myocardial tissues mechanics but increases relative severity of contractile alternans. This formulation also negatively affects the morphology and proliferation of endothelial cells which could compromise proper vessel formation and function. The inventors selected the 50/50 medium mixture for use.

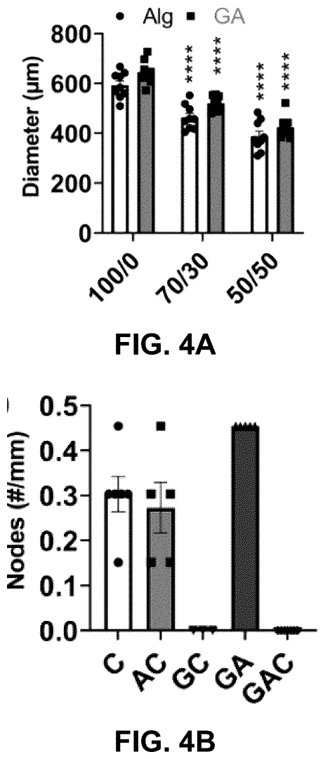

Coating solution enables local delivery of endothelial cells to channel wall. The inventors modified an alginate fiber wet spinning technique by Kant, Bare, & Coulombe, Tissue Eng. Part A, 27 (19-20), 1290-1304 (October 2021), incorporating 2.5% w/v gelatin into the 100 mM CaCl₂ crosslinking bath, generating sacrificial alginate fibers ensheathed in gelatin (GA fibers) to provide a surface for endothelial cell attachment. In considering the co-culture application of these fibers, the inventors measured diameter by incubating the fibers in 100/0, 70/30, and 50/50 media. Image analysis showed an insignificant diameter increase when alginate fibers were coated in gelatin consistent with a thin coating. An inverse relationship between EGM-2 content in medium and fiber diameter was seen despite fiber composition, with 50/50 medium resulting in the smallest diameter gelatin-alginate fibers (GA 50/50: 425±14.27 μm, p<0.0001) relative to both 70/30 (GA 70/30: 520.05±10.08 μm, p=0.0003) and control media (GA 100/0: 643.06±15.52 μm). See FIG. 4A. This pattern was present in alginate fibers as well. The decrease in fiber diameter is likely due to greater divalent cation concentration in mixed media formulations including calcium (˜1.6 mM in basal EBM-2, compared to 0.42 mM in RPMI 1640) which is involved in crosslinking alginate chains, although external protein content contributions from FBS in EGM-2 could function in equilibrium swelling.

The inventors next developed a coating solution to homogeneously seed endothelial cells on the outer surface of the gelatin-alginate fibers. Alginate fibers were positioned by embedding into the walls of PDMS molds to replicate the final EHM-casting protocol, and a variety of coating solutions were tested. A viscous hydrogel-based solution was used because seeding endothelial cells in culture medium alone resulted in the suspension dripping off the fibers before cells adhered. Seeding in a 1 mg/mL collagen solution was uneven and tended to ball up in areas forming nodes likely due to self-cohesion and slow gelation time for the coating. The inventors found that a multicomponent coating solution of 1% w/v gelatin, 0.25% w/v alginate, and 1 mg/mL collagen enabled facile and uniform coating along the length of the fiber using a micropipette. Including gelatin was used to reliably create a smooth interface with gelatin-alginate fibers and reduce node formation, while a low concentration of alginate provided rapid crosslinking with residual calcium on the gelatin-alginate fibers before longer-term gelation of the collagen to form a stable gel layer. See FIG. 4B. The inventors tested the optimized coating solution in vitro by incorporating endothelial cells at a density of 2.5×10⁷ cells/mL in coating solution, applying the seeding solution to the fibers (about 1.25×10⁵ endothelial cells total), and embedding the coated gelatin-alginate fibers in a 2 mg/mL collagen gel. Gelatin-alginate fibers were un-crosslinked and washed out the next day to create patent vessels. Culture over five days in 50/50 medium resulted in nearly full circumferential coverage and conserved potency, showing successful endothelial cell delivery and engineered vessel formation with patency maintained for at least five days in vitro.

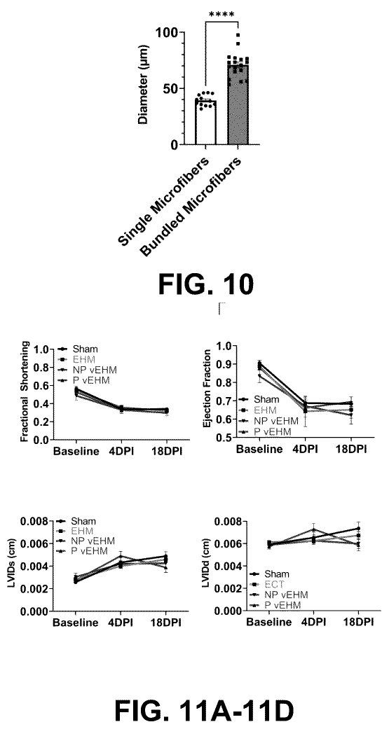

Dynamic perfusion culture of vEHMs. To develop larger constructs with perfusable vasculature for therapeutic effect in rats, the inventors scaled up the fabrication process to create 10×12 mm tissues, before compaction. The PDMS molds included a thicker wall section on one end to accommodate the insertion of a stainless steel needle for dynamic perfusion culture of vEHMs. Gelatin-alginate fibers were embedded into the walls of the mold to create an array pattern of intersecting channels and bundled collagen microfibers were embedded along the longitudinal margins of the tissue mold to provide greater structure to the tissue, improved handleability during implant, and height alignment with the perfusion needle. See FIG. 10.

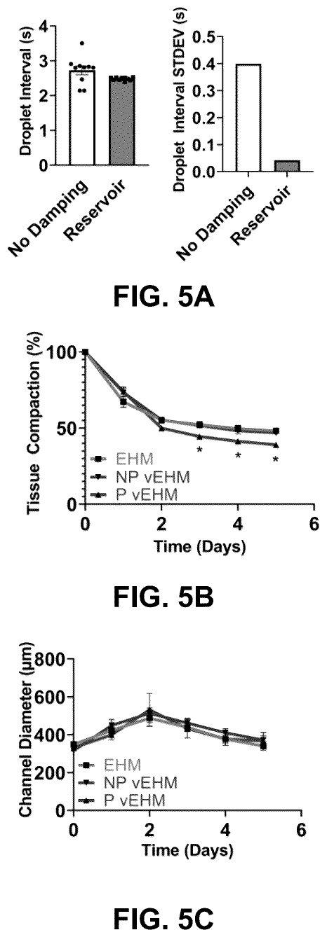

To form tissues, gelatin-alginate fibers were coated with endothelial cells (2.5×10⁷ cells/mL; vEHMs) or without endothelial cells (control EHMs) before casting a cell-collagen mixture containing hiPSC-cardiomyocytes (1.5×10⁶ cells/mL) with 5% human cardiac fibroblasts and incubated overnight. The next morning, gelatin-alginate fibers were un-crosslinked and residual alginate was washed out to establish patent vessels. After chelation, a subset of tissues with endothelialized vessels were connected to a peristaltic pump-based perfusion system for open loop recirculation of medium. The tissues were paced under 1 Hz field stimulation. A reservoir system was incorporated into the flow circuit to dampen the natural pulsatile flow of the pump and trap air bubbles to prevent embolism. Fluid velocity of the pump was determined by rotational speed of the connected stepper motors, which were controlled by an Arduino. Peristaltic pump flow was experimentally measured as 7.28 mL/hour, and average shear stress was calculated to be 3.21 dyn/cm² assuming Poiseuille flow and average vessel diameter of 400 μm. See FIG. 5A. This is a low yet physiologically relevant shear stress for endothelial cells and within reasonable range (1-10 dyn/cm²) of comparable literature. See Redd et al., Nature Commun., 10, 1-14 (2019); Ballermann, Dardik, Eng, & Liu, Kidney Int., 54, 5100-5108 (1998); Dhawan et al., Expert Rev. Cardiovasc. Ther., 8, 545-556 (2010); Debbi et al., Biomaterials. 280, 121286 (2022); Fang et al., Adv. Healthcare Mater., 9, 1-9 (2020); and Kurokawa et al., Tissue Eng. Part C Methods, 23, 474-484 (2017).

During five days of in vitro culture, significant remodeling occurred with P vEHMs compacting significantly more (39.08%±1.83%) than non-vascularized control (48.24%±1.80%, p=0.014) or NP vEHMs (46.89%±1.76%, p=0.038) See FIG. 5B. Diameter increased transiently during this time due to the un-crosslinking treatment of fibers causing swelling of the alginate chains with calcium chelation before washout. Fiber diameter re-stabilized to about 400 μm during the culture period and remained similar across groups. See FIG. 5C. Histological evaluation at five days showed endothelialized vessels in vEHMs, with elongated hiPSC-cardiomyocytes primarily located at the edges of tissues and more rounded cardiomyocytes located in the bulk of tissues where there was a lack of passive tension cues to direct alignment.

These results show robust vEHMs with stable, perfusable vasculature can be fabricated and cultured in vitro.

P vEHMs exhibit semi-conserved pattern morphology and improved engraftment.

After in vitro fabrication and assessment of vascularized engineered human myocardial tissues, the inventors evaluated engraftment onto infarcted hearts using a rat model of ischemia-reperfusion myocardial infarction. Engineered human myocardial tissues were implanted four days after infarct and cardiac function was evaluated after two weeks, with two animals excluded from analysis due to small infarcts that did not impact FS. No group (n=3-4/group) displayed significant improvements in heart function from 1D M-mode echocardiography images by calculated FS and ejection fraction (EF) at the 2-week time point despite promising trends for the P vEHM group. See FIG. 11.

The relatively short assay duration was selected to evaluate early persistent neovascularization rather than to evaluate contractile function endpoints. Hearts were then harvested and underwent ex vivo OCT and microCT imaging before sectioning for histological evaluation. During Microfil® perfusion, the inventors observed a distinct vessel array reminiscent of the original pattern geometry was visible in one of three hearts implanted with P vEHMs.

Engineered human myocardial tissues and NP vEHM implants were distinctly paler in color than P vEHMs, suggesting greater intra-implant perfusion in the latter group.

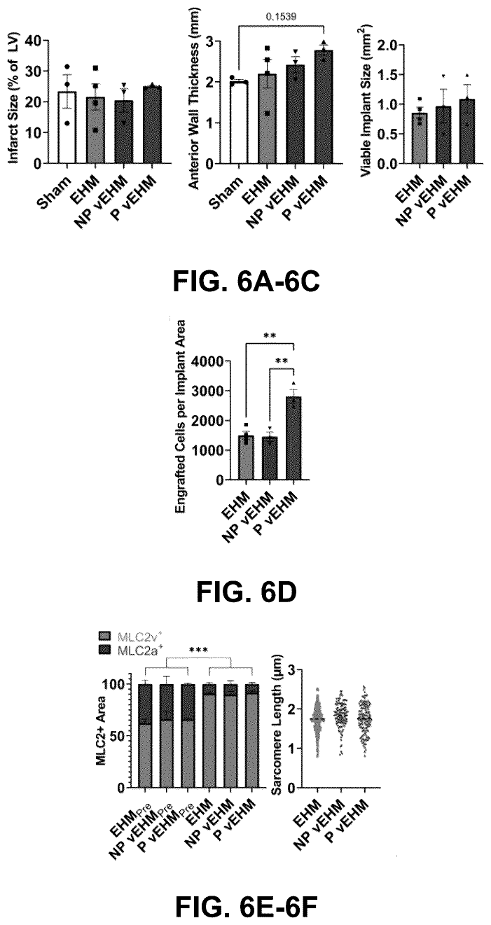

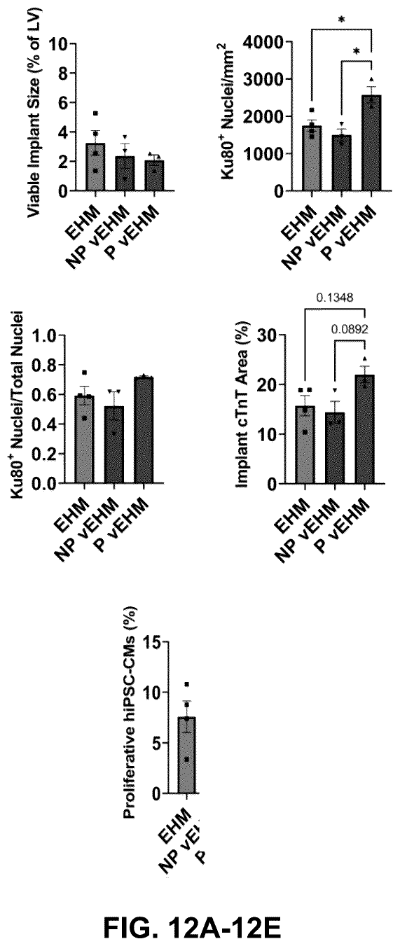

Morphometric analysis of hearts from picrosirius red-fast green stains indicated comparable infarct size and anterior wall thickness excluding implants. See FIGS. 6A-6B. Implants were visible and often surrounded by vascularized adhesion tissue from the chest wall. Viable implant size was measured by measuring the area of live hiPSC-cardiomyocytes from immunohistochemical stains of Hu-Ku80, but no significant difference was found between groups (EHM: 0.86±0.09 mm², NP vEHM: 0.97±0.29 mm², P vEHM: 1.09±0.24 mm²). See FIG. 6C. See also FIG. 12A.

Nuclei measurement showed significantly greater human cell density in P vEHMs (see FIG. 12B) and increased engraftment when normalized by implant area (2806±239.3 Hu-Ku80+ nuclei/mm²) as compared to engineered human myocardial tissues (1499±132.0 Hu-Ku80+ nuclei/mm², p=0.0024) and NP vEHMs (1452±154.1 Hu-Ku80+ nuclei/mm², p=0.0029). See FIG. 6D. Total intra-implant cell nuclei were not different across groups (see FIG. 12C), but evaluation of co-stained cTnT+ area in implants showed trends towards increased hiPSC-cardiomyocyte area in P vEHMs (22.01±1.68%) that did not reach significance relative to NP vEHMs (14.41±2.19%, p=0.13) or control (15.75±2.03%, p=0.089). See FIG. 12D. This metric could be confounded by relative hiPSC-cardiomyocyte compaction and remodeling with the host across groups. The inventors then assessed hiPSC-cardiomyocyte proliferation by Hu-Ki67 staining. See FIG. 12E. The inventors found no differences across groups. These results suggest that the difference in engraftment of P vEHMs is primarily due to greater survival of hiPSC-cardiomyocytes, rather than proliferation of hiPSC-cardiomyocytes, engraftment of endothelial cells and human cardiac fibroblasts, or infiltration of host cells.

Ventricular isoform expression of MLC2 was greater in the implant groups (89.68%-91.65% MLC2v+ by area) compared to in vitro pre-implantation control tissues (62.16%-65.75% MLC2v+ by area, all p<0.001), although there were no differences between implant groups. See FIG. 6E. Measurements of sarcomere length in implants were not significantly different between implant groups (EHM: 1.73±0.02 μm, NP vEHM: 1.82±0.03 μm, P vEHM: 1.75±0.03 μm). See FIG. 6F. This indicates that engraftment promoted maturation as assessed by MLC2v isoform switching for the tissues. These data provide evidence that perfused, patterned engineered vessels can be conserved and perfused in vivo by inosculation. The in vitro perfusion of engineered vessels improved hiPSC-cardiomyocyte survival and engraftment density.

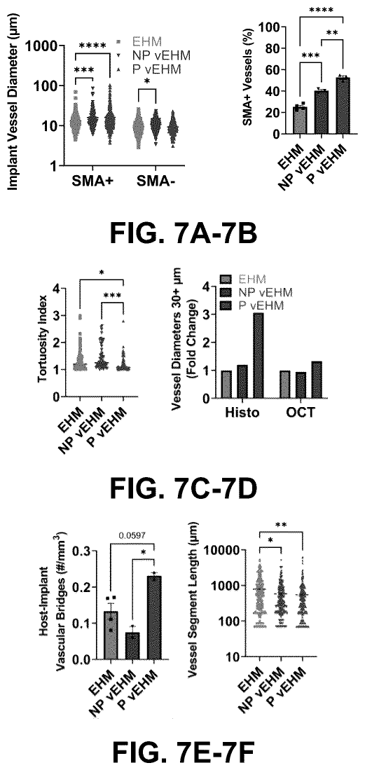

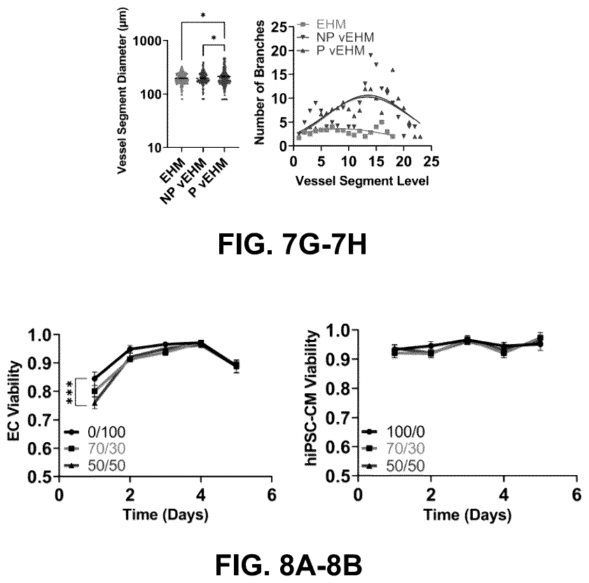

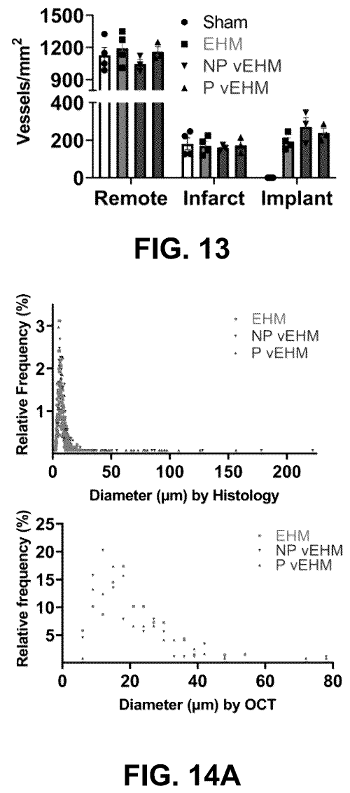

P vEHMs develop larger and more hierarchically organized vasculature. To identify how perfused, patterned vessels in EHMs affect vascular development in vivo, the inventors first performed histological measurement of pan-vascular isolectin B4 and Hu-Ku80 in the remote, infarct, and implant regions of each group, although no significant differences were found. See FIG. 13. The inventors next performed histological stains for isolectin B4 and SMA (cross-registered to Hu-Ku80 stains to only measure vessels in areas of viable implant) and found significantly increased diameters of SMA+ vessels in NP vEHMs (15.89±0.72 μm, p=0.0004) and P vEHMs (16.06±0.71 μm, p<0.0001) compared to control EHMs (13.16±0.52 μm). See FIG. 7A. This was accompanied by a greater frequency of SMA+ vessels in P vEHMs (52.69±1.66%, p<relative to EHM and p=0.0017 relative to NP vEHM), and to a lesser extent, NP vEHMs (40.46±1.06%, p=0.0003) as compared to EHMs (25.31±1.41%). See FIGS. 7A-B. Co-staining for human-specific CD31 (Hu-CD31) with isolectin B4 showed two distinct phenotypes in NP vEHMs and P vEHMs. Human endothelial cells either appeared in capillary-like sprouts in NP vEHMs or associated with vessel walls, potentially indicating a patterned vessel. This latter phenotype was present in P vEHMs, but no isolated human endothelial cell sprouts were found.

While Microfil® overlays were captured via light microscopy where possible, the material was not reliably maintained in vessels, particularly those with larger lumens, during histological processing after microCT scanning.

Some Microfil®-filled lumens did contain Hu-CD31+ cells in NP vEHMs and P vEHMs, providing evidence of functional inosculation and perfusion of patterned vessels in both groups and/or evidence of human endothelial cells incorporating into newly formed vessels of rat origin.

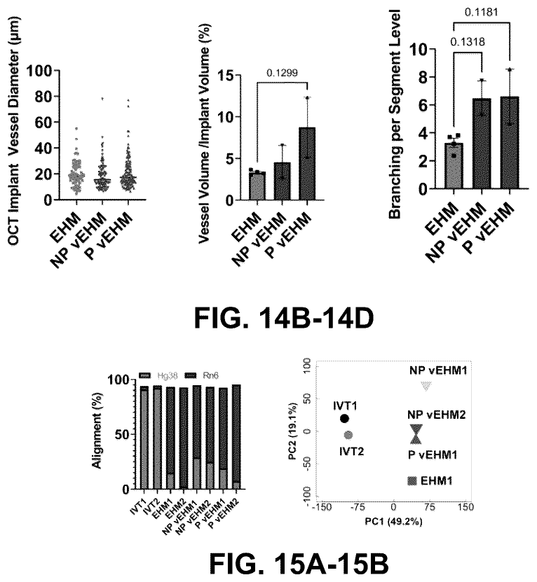

To better understand the 3D architecture and perfusability of intra-implant vasculature, the inventors performed OCT imaging of the remote, infarct, and implant regions for each group. Cardiac microangiograms were acquired using 20% Intralipid solution as contrast to visualize hierarchically branched vascular architecture down to the capillary scale. Acquisition at border and infarct regions displayed increasingly tortuous vasculature and collaterals, and less resolvable capillary perfusion. EHM microangiograms exhibited variable perfusability, often with gaps that appeared lowly perfused and/or highly tortuous (1.35±0.04 tortuosity index). Microangiograms of vEHMs showed greater perfusable vessels with typically larger sizes, with significantly less tortuosity in P vEHMs (1.2±0.03 tortuosity index) relative to NP vEHMs (1.46±0.06 tortuosity index, p=0.0002) and control EHMs (p=0.025). See FIG. 7C. The inventors measured diameter from OCT microangiograms and, akin to histological assessments, found trends of larger vessels in NP vEHMs and P vEHMs. See FIG. 7D. See FIG. 14. But this did not reach significance relative to control.

Following OCT microangiography, the hearts were perfused with Microfil® to enable microCT-assisted reconstructions of the cardiac vasculature. Three hearts were excluded from further analysis due to poor Microfil® perfusion. The implant regions were isolated for vascular reconstructive analysis by manual segmentation and cross-registration with histological sections. The inventors measured host-implant vascular bridges and observed a significant increase in P vEHMs (0.231±0.010 bridges/mm³) relative to NP vEHMs (0.075±0.016 bridges/mm³, p=0.018), and a trending increase relative to control EHMs (0.133±0.022 bridges/mm³, p=0.059) that did not quite reach significance. See FIG. 7E. The total intra-implant vascular volume did not reach significance across groups. See FIG. 14C. But there was significantly decreased vessel segment length in endothelialized vEHMs (NP vEHM: 583.6±47.09 μm, p=0.036; P vEHM: 546.2±48.98 μm, p=0.007) relative to control EHMs (787.6±72.39 μm). See FIG. 7F.

Vessel segment diameter was significantly increased in P vEHMs (213.7±5.61 μm) relative to NP vEHMs (196.6±4.01 μm, p=0.024) and control (196.5±3.47 μm, p=0.039). See FIG. 7G. The inventors also observed trends towards increased branching per segment level in endothelialized vEHMs, pointing to greater vascular hierarchy than EHMs. See FIG. 7H. See FIG. 14. These results show patterned vasculature enables the development and maintenance of stable, large diameter vessels with greater branching than control tissues, and in vitro perfusion enables a more stable endothelial cell phenotype with less human endothelial cell migration away from patterned vessels in vivo and greater hierarchical organization.

Exploratory bulk RNA sequencing of explanted vEHMs suggests perfusion influences hiPSC-cardiomyocyte development and contractility. The inventors further assayed the effects of implantation and patterned, perfused vasculature on hiPSC-cardiomyocyte genotype, as a precursor to phenotype shifts, through exploratory bulk RNAseq analysis of explanted tissues. At the two-week timepoint, a subset of implanted tissues was manually debrided from the epicardial surface and processed for bulk RNA isolation and sequencing. Alignment analysis produced an expected heterogeneous mixture of human and rat-derived reads in explanted tissues, and by comparison almost 100% human-aligned reads in in vitro control tissues. See FIG. 15A.

Principle component analysis plots showed stratification by condition with implants expectedly segregated from in vitro control tissues primarily by principal component 1 (representing 49.2% of differences) due to contributions of the local in vivo microenvironment, and implant groups further separated across principal component 2, representing an additional 19.1% of differences. See FIG. 15B. Normalized counts for genes of interest in cardiomyocyte development, maturation, and vascularization were plotted for each condition, showing trends of upregulation of developmentally regulated genes involved in myofibril assembly and cardiac contractility (MYH1, MYH3, MEF2C, XIRP2, MYL2, MYL7) and calcium handling (ATP2A1, ATP2A2). Vascularization panels for human endothelial cells showed trends for downregulation of several growth factors and receptors relative to in vitro non-perfused controls (VEGFA, FGF11, IGF1R, PDGFRA, PDGFRB, FLT1), suggestive of reduced human vascular signaling in vivo that follows turnover of human endothelial cells for host rat endothelial cells.

To further explore differential regulation between groups, the inventors performed a preliminary analysis of DEGs between explant groups assuming equal sample variance to account for poor sample sizes owing to samples excluded due to low alignment to the human genome (final n=1-2/group). In this analysis, only 9 and 13 genes were identified as differentially expressed for P vEHM vs EHM and P vs NP vEHM, respectively. Subsequent GO analysis produced terms involved in myofilament development, calcium handling, and contractility, implying a function for in vitro perfusion of vEHMs to amplify the in vivo development of hiPSC-cardiomyocytes. The RNAseq results implicate a function for vascularization and perfusion in the biological development of hiPSC-cardiomyocytes, providing some support for a co-vascularization strategy for remuscularization by implanted vEHMs is beneficial for holistic and robust cardiac regeneration. The RNASeq analyses of explanted cardiac tissue represents a large leap in the field. Only one group recently published bulk RNA sequencing of intramyocardially injected hiPSC-CMs in a rodent model of I/R myocardial infarction. Marchiano et al. Cell Stem Cell, 30, 741 (2023). To the best of the inventors' knowledge, this is the first report on bulk RNA sequencing of explanted cardiac tissues from a multi-species preclinical model.

Discussion. In this EXAMPLE, the inventors showed a facile method for fabricating and culturing EHMs with patterned vasculature under static or dynamic perfusion conditions. the results indicate that in vitro perfused engineered vessels in vEHMs improve in vivo hiPSC-cardiomyocyte survival and engraftment density; implant vascular organization and perfusion; and potentially also hiPSC-cardiomyocyte contractile development as suggested by RNAseq analysis. Other vessel patterning techniques require expensive equipment and/or materials that cannot be autoclaved or otherwise easily sterilized. Mirdamadi et al., ACS Biomater. Sci. Eng., 6, 6453-6459 (2020); Kinstlinger et al., Nature Protoc., 16, 3089-3113 (2021). These methods provide a flexible foundation for creating perfusable and implantable vEHMs in standard 6-well tissue culture plates. These methods use equipment and biomaterials that can be fabricated in-house using the open-source plans or otherwise commercially available materials and technologies.

These results directly support the use of perfused, endothelialized vessels in engineered human cardiac implants to improve both the vascular bed and the engrafted hiPSC-cardiomyocytes.

The success of hiPSC-cardiomyocyte engraftment after engineered human myocardial tissue implantation onto the infarcted left ventricle is a function of multiple design parameters. The relatively immature metabolic profile of terminally differentiated hiPSC-cardiomyocytes could help with survival due to greater resistance to ischemia such as that experienced at implantation. Peters et al., Stem Cells Transl. Med., 11, 1040-1051 (2022). The cardiac/endothelial medias tested in this assay are devoid of fatty acids. They do not help with metabolic maturation. But the selected 50/50 culture medium enabled some structural maturation of cardiomyocytes. See Dhahri et al., Circulation (2022); Feyen et al., Cell Rep., 32, 107925 (2020). The increased calcium in growth medium (EBM-2 basal media has ˜1.6 mM Ca) during in vitro culture. Minor & Coulombe, Stem Cells Transl. Med., 1-10 (2022). Growth factors include VEGF and IGF. See Shi et al., Biochim. Biophys. Acta—Mol. Cell Res., 1853, 1154-1164 (2015); Rottbauer et al., Genes Dev., 19, 1624-1634 (2005); Funakoshi et al., Nature Commun. 12 (2021); Laflamme et al., Nature Biotechnol. 25, 1015-1024 (2007); Munarin, Kant, Rupert, Khoo, & Coulombe, Biomaterials, 251, 120033 (2020); and Rupert & Coulombe, Stem Cells Int., 2017 (2017). The improvements in hiPSC-cardiomyocyte engraftment were seen in P vEHMs, suggesting that endothelial cells in the channels without perfusion as in NP vEHMs was not enough to maintain hiPSC-cardiomyocyte density upon implantation, but could be rescued by in vitro perfusion. This metric of hiPSC-cardiomyocyte engraftment density was higher than comparable assays despite similar graft sizes. Munarin, Kant, Rupert, Khoo, & Coulombe, Biomaterials, 251, 120033 (2020); Brady et al., APL Bioeng. 4, 016105 (2020); Redd et al., Nature Commun., 10, 1-14 (2019); Gerbin, Yang, Murry, & Coulombe, PLoS One, 10, 1-20 (2015); and Weinberger et al., Sci. Transl. Med., 8, 363ra148-363ra148 (2016). This was independent of proliferation.

It is important to investigate what enables patterned vessels to be functionally used by the regenerating vasculature through the process of inosculation. Some patterned vessels could not be reliably identified owing to remodeling, endothelial cell turnover, and limits of cardiac vascular imaging. Evidence of residual Microfil® in Hu-CD31+ vessels of NP vEHMs and P vEHMs suggests that channel endothelialization helps with inosculation and perfusion of patterned vessels. Up-stream arteriogenic processes could be required of host vasculature to help with greater blood flow across the vascular bridges from host to implant. In vitro perfusion could have helped with a more stable endothelial cell phenotype that was less prone to migration away from pattered channels when implanted in vivo, as shown by the lack of isolated human endothelial cells in P vEHMs compared to NP vEHMs. Histological staining showed greater numbers of SMA+ vessels in P vEHMs of the diameters compared to other groups. This staining indicates endothelialization and particularly in vitro perfusion can help with the process of vessel stabilization and maturation of both inosculated vessels and angiogenic branches by recruitment of SMA-expressing stromal cells, such as smooth muscle cells, fibroblasts, and pericytes, resulting in faster and more stable implant perfusion post-implantation. Brady et al. showed that their non-perfused patterned microvessels were disrupted when implanted on the rat heart, resulting in isolated endothelial cells reminiscent of one of the vascular phenotypes of human endothelial cells seen in NP vEHMs. See Brady et al., Sci. Rep. 13, 1973 (2023). Single cell and bulk RNAseq assays of endothelial cells exposed to flow showed differential regulation of genes suggesting a more quiescent and stable phenotype than no-flow controls. Helle, Ampuja, Antola, & Kivela, Frontiers Physiol., 11, 1-13 (2020). Inosculation is further shown by the conserved pattern geometry visually confirmed in one out of three vEHM-implanted hearts perfused with Microfil®. Inosculation may have occurred in the other two vEHM-implanted hearts. The macroscopic visual assessment and microCT reconstructions were not conclusive, despite Microfil® within Hu-CD31-positive vessels by 2D immunohistochemical analysis. Inosculation of some vessels may have been obscured by in vivo remodeling of human endothelial cell-lined channels into perfusable vessels or from unresolved thrombosis resulting in only partial perfusion of the patterned vessel array. See White et al., Tissue Eng. Part A, 20, 2316-2328 (2014); Ben-Shaul, Landau, Merdler, & Levenberg, Proc. Natl. Acad. Sci., 201814238 (2019).

Others reported that larger diameter endothelialized vessels (>400 μm) can help with vessel retention, inosculation, and perfusion in vivo, although often with remodeling to a smaller diameter. Mirabella et al., Nature Biomed. Eng., 1, 0083 (2017); Debbi et al., Biomaterials, 280, 121286 (2022); Szklanny et al., Adv. Mater. 33, 2102661 (2021). Smaller diameter microvessels (˜50-200 μm) show variable success in metrics of inosculation and perfusion. See Sun et al., Sci. Transl. Med. 12, 1-11 (2020); Brady et al., APL Bioeng. 4, 016105 (2020); Redd et al., Nature Commun., 10, 1-14 (2019); Brady et al., Sci. Rep. 13, 1973 (2023); and Baranski et al., Proc. Natl. Acad. Sci. 110, 7586-7591 (2013). Direct comparison of 200 μm and 400 μm patterned vessels by Mirabella et al. showed the smaller vessels did to rescue perfusion in a hind-limb ischemia model, suggesting initial patterning diameter remains an important consideration in revascularization. Mirabella et al., Nature Biomed. Eng., 1, 0083 (2017). Channels alone without endothelial cells, as in control EHMs, can directionally instruct host vessel ingrowth. Channels alone without endothelial cells provide an indirect in vivo benefit in revascularization. Tang et al., ACS Biomater. Sci. Eng., 6, 1476-1486 (2020); Jiang et al., Mater. Sci. Eng. C., 126, 112178 (2021); and Parkhideh et al., Biomater. Sci. (2023).

The inventors did not positively identify non-vascularized EHM channels reliably at explant due to remodeling and a lack of tracer in the channels. Others, including Mirabella et al. showed no improvements in perfusion of hind limbs with non-endothelialized channels. Mirabella et al., Nature Biomed. Eng., 1, 0083 (2017). Szklanny et al. showed that non-endothelialized channels (1500 μm diameter) were incapable of effective long-term perfusion even when directly anastomosed to the femoral artery of rats, and even reported some animal mortality in this group, possibly from a thrombus that formed in the channel and dislodged. These results support the idea that non-endothelialized channels are not experiences by the host as true vessel lumens for inosculation and act via indirect mechanisms to guide angiogenic sprouts. Szklanny et al., Adv. Mater. 33, 2102661 (2021).

In contrast to patterning methodologies, reliance on vascular self-assembly via bulk seeding of endothelial cells alone seemingly provides little inosculative perfusion (Redd et al., Nature Commun., 10, 1-14 (2019)) or penetration into implanted tissue (Baranski et al., Proc. Natl. Acad. Sci. 110, 7586-7591 (2013)) without co-seeding with stromal cells (Schaefer et al., J. Tissue Eng. Regen. Med., 12, 546-556 (2018); White et al., Tissue Eng. Part A, 20, 2316-2328 (2014)) or combined with patterned vessels of any scale (Redd et al., Nature Commun., 10, 1-14 (2019); Debbi et al., Biomaterials, 280, 121286 (2022). The prior work showed significant benefits on vessel development of angiogenic growth factor release from microspheres embedded in hiPSC-cardiomyocyte implants. Munarin, Kant, Rupert, Khoo, & Coulombe, Biomaterials, 251, 120033 (2020). This work suggested that engineered paracrine signaling may circumvent the need for bulk-embedded endothelial cells. These results are useful to explore a dual microvascular and meso-vascular approach to implant revascularization using growth factor release and arteriole-scale vessels, and to evaluate how autocrine and paracrine endothelial cell signaling both within implants and between host and implant endothelial cells differentially modulate new vessel formation.

To assess small vessel perfusion in a thicker volume of tissue, OCT imaging provides a state-of-the-art imaging modality that can be applied to the heart ex vivo. OCT is not widely available in preclinical assays. Few other groups have performed OCT imaging of the heart and of implanted vascular tissues. See Qin et al., Phys. Med. Biol., 61, 7536-7550 (2016); Redd et al., Nature Commun., 10, 1-14 (2019). One other group published OCT images on implanted cardiac tissue as proof-of-concept that patent vessels can be imaged. Brady et al., APL Bioeng., 4, 016105 (2020). The high fidelity microangiograms achieved in this specification allow for quantitative comparisons across implant groups. The inventors showed greater in vivo vessel organization could be achieved by in vitro perfusion of patterned vessels to avoid tortuous vessel development reminiscent of collateral vessels in the infarct and border regions. Capillary level perfusion is difficult to image in the deeper injury region of the left ventricular wall and epicardial implant, and often complicated due to artifacts of the fixed vessels (with reduced compliance), perfusion pressures, capillary collapse, adhesion tissue leakage from explant, operator skill, or other reasons, making measurement of flow rate and vascular resistance difficult.

Intra-implant perfusion velocity using dynamic light scattering OCT (Lee, Wu, Jiang, Zhu, & Boas, Opt. Express. 20, 22262 (2012)) or live OCT acquisition could advance the understanding of blood flow dynamics in the implant and infarct regions of the post-myocardial infarction regenerated heart with enough contrast and correction of motion artifacts from the contracting heart.

As vascular regeneration aims to support larger and thicker tissues in translational applications, engineering vascular network hierarchy requires development of whole-implant and even whole-organ quantitative vessel analyses in three dimensions. The inventors published on the use of microCT for 3D vascular reconstruction and analysis. Munarin, Kant, Rupert, Khoo, & Coulombe, Biomaterials, 251, 120033 (2020). In this EXAMPLE, the inventors implemented a vessel reconnection algorithm based on a minimum-work principle of Jiang et al., J. Invest. Dermatol. (2011), pp. 528-536, to semi-automate the processing steps and provide more accurate metrics of the vessels in implanted tissues. This advanced analysis provides greater insight to intra-implant vessel hierarchy.

Despite no differences in lumen density across conditions in either the infarct or implant regions (FIG. 13) in the histological assessment, the inventors saw an increased frequency of larger diameter vessels in vEHMs relative to capillary-scale vasculature (FIG. 7B). the microCT analysis of vessel diameter corroborated histological measurements and showed greater branching, and interestingly more host-to-implant vessel bridges in P vEHMs (FIG. 7E-H), together suggesting that a more hierarchical vascular bed formed with endothelialized vessels and was most functionally improved by in vitro perfusion of vEHMs.

Because of the computationally costly large data sets generated with whole heart reconstruction, the inventors analyzed the implant volume at a scan resolution of 50 μm to capture a range from large vessels to meso-diameter arterioles. An artifact of this methods was several single-voxel “vessels” with a reported diameter and length of 50 μm which were eliminated from the analysis to provide a more accurate measurement of vessel hierarchy. The resulting branching analysis showed a negative parabolic relationship between vessel segment level and number of branches. While expected from a native vascular network of arteries that branch into capillaries and coalesce back into veins, the resolution limits of the analysis suggest this relationship is rather an artifact of branch “termination” below 50 μm in diameter. Use of the highest possible scanning resolution (5-10 μm) requires both sub-sampling implant volume and optimization of Microfil® perfusion to ensure perfusion of the vascular tree, although the vascular volume is comparable to published performing protocols in skeletal and cardiac muscle. Redenski, et al., ACS Biomater. Sci. Eng., 8, 232-241 (2022); Weyers et al., PLoS One, 15, e0227780 (2020). Intra-implant vasculature visualized by OCT and microCT data and vascular bridges indicate appropriate perfusion through implants. Identification of arterial and venous vasculature to assess relative perfusion efficiency can be pursued by co-registration of OCT microangiograms and velocity maps with microCT data sets if appropriate landmarks (such as multiple host-implant bridges) can be identified for 3D registration. Further development of imaging techniques should enable dynamic longitudinal assays and flow measurement of remodeling vasculature.

Limited literature examines the impacts of in vitro perfusion and in vivo vascularization on hiPSC-cardiomyocyte structural and functional maturation. Sun et al., Sci. Transl. Med. 12, 1-11 (2020); Gomez-Garcia, Quesnel, Al-attar, Laskary, & Laflamme, Semin. Cell Dev. Biol. (2021); Cruz-Moreira et al., Biotechnol. Bioeng., 118, 3128-3137 (2021). But developmental evidence suggests cardiac maturation is intimately coupled with coronary growth. Karra et al., Proc. Natl. Acad. Sci., 115, 8805-8810 (2018); DeBenedittis et al., Development, 149 (2022). To show that co-revascularization with remuscularization by vEHM implantation improves engrafted hiPSC-cardiomyocyte development and maturation, the inventors pursued bulk RNAseq analysis on explanted EHMs after two weeks in vivo to explore early genotype shifts.

The preliminary analysis with equal variance assumed showed upregulation of genes related to cardiac development in vEHMs relative to control. Although not conclusive given the sample size and analysis methodology, the preliminary results show promise in pursuing RNAseq to elucidate mechanisms of vascular-driven growth and maturation. Some groups have shown RNAseq analyses of cardiomyocytes in 2D and 3D in vitro tissues for cardiac applications. Redd et al., Nature Commun., 10, 1-14 (2019); Tiburcy et al., Circulation, 135, 1832-1847 (2017); and Feyen et al., Cell Rep., 32, 107925 (2020). Other groups have shown whole-heart bulk and single-cell RNA sequencing. Quaife-Ryan et al., Circulation, 136, 1123-1139 (2017); McLellan et al., Circulation, 142 1448-1463 (2020); Wang et al., Nature Commun., 11, 2585 (2020).

The inventors can optimize isolation procedures to ensure high quality data for bulk or single cell/nuclei RNAseq and pursue multi-species alignment deconvolution for advancing assessment of host-implant heterocellular vascular interactions and their contributions to cardiac regeneration. See Kluin et al., BMC Bioinformatics, 19, 366 (2018).

The inventors have pursued mechanistic understanding of regeneration at multiple time points, such as how hiPSC-cardiomyocyte development and maturation is facilitated by inosculation and what the contributions of a co-revascularization strategy with remuscularization are for long-term in vivo maturation and durable heart regeneration.

This EXAMPLE examined specific effects of mixed media on hiPSC-CMs and endothelial cells in 2D and 3D. While negative effects on endothelial cell proliferation were unsurprising as EGM-2 media is optimized specifically for endothelial cell culture in vitro, the massive improvements seen in hiPSC-CM contractility and tissue structure were unexpected. Despite being the gold standard media for hiPSC-CM differentiation and culture, RPMI B27 was originally created for hippocampal neuronal culture and does not effectively recapitulate a CM environment. B27 media has high glucose (10 mM), low calcium (0.42 mM), and no fatty acids for beta oxidation, but the in vivo CM microenvironment is relatively low in glucose, high calcium, and high reliance on fatty acid oxidation for ATP generation. A 50/50 mix of B27 and EGM-2 contributes a slightly higher calcium concentration (EBM-2 basal media has 1.6 mM Ca2+), and growth factors including VEGF, bFGF, IGF, and EGF. The inventors have shown that 3D culture of ECTs in physiological calcium levels results in improved calcium handling ability and contractility. The inventors showed that incorporation of microparticles releasing VEGF resulted in increased tissue stiffness but not contractility at four weeks, suggesting a function in tissue remodeling. IGF is a well-known factor in cardiomyocyte biology, contributing to multiple pathways including fetal proliferation, hypertrophy, and ischemic protection. Histology of the hiPSC-CMs in 2D and 3D showed greater sarcomere development and hypertrophy when cultured in mixed media conditions relative to B27 alone.

Dynamic perfusion culture induced greater compaction than NP vEHMs or control EHMs, suggesting greater remodeling of the tissues. The estimated shear rate of 3 dyn/cm² set in the perfusion assays is relatively lower than the average shear rates in human coronary arteries (about 5-12 Pa), though within reasonable limits of 1-10 dyn/cm² in comparable literature.

In pilot assays, greater flow rates and shear stresses were deleterious to the structure of the entire tissue. Lower shear stresses in arteriole-scale vessels such as those used in this EXAMPLE have been atherogenic in vivo, though in vitro fabricated vessels displays a naiver phenotype than in vivo vasculature.

The cellular composition of vEHMs functioned in constraining the geometry of the tissue. This compaction also introduced several design compromises when introducing large, perfusable channels into the tissue. One such trade-off was the use of internal post geometries which can improve hiPSC-CM alignment and contractile potential. Tiburcy et al. showed peak active force was reached when cardiac tissues were composed of about 60% cardiomyocytes. Inclusion of more fibroblasts would increase compaction and lead to the risk of greater channel closure during culture time. Because of the increased size and relative lack of tension cues in the bulk of engineered human myocardial tissues, hiPSC-CMs adopted a more rounded morphology compared to elongated and aligned morphology at the edges.

Implantation of perfused vEHMs onto the infarcted left ventricle of rodent hearts showed improved patch engraftment, reduced vessel tortuosity, and improved hiPSC-CM contractile development. the inventors also noted a trend towards increased free wall thickness, though this metric did not reach significance. The inventors excluded added thickness from the implant itself because this metric would be inflated by variable levels of adhesion tissue that deposited between myocardial infarction and implant surgery, as well as over the two week analysis. The lack of engrafted hiPSC-CMs in NP vEHMs was surprising, suggesting that channels alone were not enough to prevent hiPSC-CM death upon implantation but could be rescued by in vitro perfusion, which may be because of early inosculation and perfusion of the tissue. the measurement showed similar viable graft size and even higher engraftment than comparable analyses. Some analyses have shown that incorporating microchannels into scaffolds improves host tissue ingrowth and directed vascular formation, suggesting that non-endothelialized channels still provide an indirect in vivo benefit in revascularization.

In examining vascular development, implanted tissues developed similar numbers of vessels despite patterned channel condition. Vessel measurement similarly was not different from that in infarct regions. improved vessel diameters in NP vEHMs and P vEHMs with some evidence of residual Microfil® in Hu-CD31+ channels suggested that channel endothelialization and helped with inosculation and perfusion of patterned channels as well as arteriogenic processes of host vasculature. Increased frequency of larger diameter vessels relative to capillary-scale ones could explain the non-significant results in vessel measurement. Redd et al. showed seeding endothelial cells into the bulk of constructs containing patterned vessels improved graft perfusion and co-implanted hiPSC-CM engraftment. Previous analyses showed the benefits of angiogenic growth factor release from microspheres embedded in implants.

OCT imaging provided insight into intra-implant vascular structure that could not otherwise be adequately captured in histological images. OCT is a relative new technique and few other groups have performed OCT imaging of the heart and of implanted vascular tissues. These results show greatly improved image fidelity and comparisons across implant groups with differing vascularization conditions. The inventors found more organized vasculature in P vEHMs compared to the relatively tortuous vessels in EHM and NP vEHMs, which was reminiscent of vessel collateralization in the infarct and border regions. It was difficult to image perfused capillaries in the injured and implant regions of the heart. It is unclear to what extent this was due to lower perfusion pressures (50-60 mmHg), relatively low vessel density, and capillary collapse. Adhesion tissue from the chest wall created an additional obstacles during imaging as dissecting this tissue away to prepare the heart for OCT acquisition resulted in occasional vascular leakages that obscured imaging capabilities.

Because of the high alignment to the rat genome, the inventors ran DGE and GO term analysis for each sample to explore host cell contributions to implant vascularization. The results noted upregulation of genes suggesting red blood cells (HBB, HBA, LGALS5) and “contaminating” rat cardiomyocytes (TNNT3, TNNI3, RYRI). These results may be due to issues with sampling of host rat cells. The inventors tried to reduce collection of non-implant cells, e.g., underlying myocardium and adhesion tissue, which could have resulted in inconsistent rat cell population collection.

The inventors developed a flexible in vitro system for fabrication and dynamic perfusion culture of vEHMs. The inventors report the structural and functional effects of mixed-media culture on human endothelial cells and hiPSC-CMs. Implantation of vEHMs in a rodent model of ischemia-reperfusion myocardial infarction showed that endothelialized channels both inosculated with host vasculature and resulted in increased intra-implant vessel diameter. in vitro perfusion improves hiPSC-CM engraftment, organization of vasculature for efficient hierarchical perfusion, and upregulation of genes related to cardiomyocyte development and maturation. This EXAMPLE provides a strong foundation for the biomedical art to understand the design criteria for fabricating complex heterocellular vEHMs in superior culture conditions. The benefits of vasculature on engineered tissue survival and maturation in vivo for therapeutic remuscularization of the heart after myocardial infarction.

The results in this EXAMPLE also show the differentiation, expansion, and maturation of human induced pluripotent stem cell-derived cardiomyocytes (hiPSC-cardiomyocytes) using a multi-stage biphasic Wnt-modulation protocol. Vascularized engineered human myocardial tissues (vEHMs) are then formed by mixing hiPSC-cardiomyocytes and 5% human primary cardiac fibroblasts into a collagen/hydrogel. Sacrificial gelatin-alginate fibers coated with endothelial cells are embedded in cardiac tissues to pattern patent, arteriole-scale channels 400 μmin diameter. vEHMs are cultured under dynamic perfusion conditions before implantation for two weeks to analyze inosculation, revascularization, and remuscularization of the heart.

Immunohistochemical staining showed engraftment of hiPSC-cardiomyocytes in the presence of patterned, perfused vasculature and inosculation with host vasculature. RNA sequencing further shows upregulation of genes related to cardiac contractility and electrophysiology in perfused vEHMs relative to control.

The establishment of engineered cardiac tissues consisting of robustly perfused implanted hiPSC-cardiomyocyte tissue by way of prevascularization using arteriole-scale vessels should help with greater patch engraftment and functional maturation. The inventors accomplished the formation of engineered human myocardium (EHM) containing perfused arteriole-scale vessels and show their robust engraft onto infarcted myocardium, improved perfusion, and maturation of engrafted hiPSC-cardiomyocytes.

### Example 2

### Translational Research

Leading translational research in large animal analyses and upcoming clinical research involving cellular therapeutics with hiPSC-CMs such as the BioVAT heart failure trial (NCT04396899) and HECTOR trial (NCT05068674) cannot as yet determine true engraftment efficiency or its impact on long-term function due to a lack of focus on a vascular part to their analyses. Higher-ordered vascular organization is incompatible with intramyocardial injection-based therapeutics. The only option being co-injection of endothelial cells or other microvessel-based sources. Even some epicardial patch-based methods used internal post geometries to direct internal tissue alignment, allowing for greater contractile alignment but similarly preventing vascular design considerations. Current analyses have eschewed this design format to accommodate larger-order vascular channels for robust prevascularization, though its effects on bulk cellular alignment in vitro. The literature has shown the benefits of a patterned vasculature on patch survival and engraftment, even if only transiently present.

As the cardiovascular tissue engineering field advances towards translational implementations of hiPSC-CM-based therapeutics, several barriers remain to be addressed. Safety is a cornerstone of phase 1 and phase 2 trials, and ongoing trials typically center primary outcomes around incidence of adverse events or arrhythmias (e.g., NCT04982081, NCT03763136, NCT04696328). Pre-clinical research, particularly at the large-animal scale, thus focuses primarily on hiPSC-CM contractile maturation, electrical coupling, and arrhythmia susceptibility. As later waves of clinical trials begin to examine clinical efficacy more rigorously through functional or surrogate endpoints for efficacy (including ongoing trials, e.g., NCT05068674, NCT04396899, NCT03759405, and NCT04696328), the importance of vascular contributions to hiPSC-CM engraftment, maturation, This EXAMPLE shows the function of revascularization co-therapy with remuscularization and is visionary in leveraging the host vascular response to improve hiPSC-CM engraftment. This EXAMPLE provides the infrastructure for large animal and clinical analyses in determining the effect of in vitro prevascularization in functional dosing analyses.

The aortic ring assay provides an opportunity for further study of vEHM inosculation in a controlled in vitro model. See Kant, Bare, & Coulombe, Tissue Eng. Part A, 27 (19-20), 1290-1304 (October 2021). Although the aortic ring remains the standard for determining angiogenic response to a stimulus, harvested vessels show distinct angiogenic phenotypes dependent on the location they were harvested from, e.g., carotid or femoral artery. For applications in cardiovascular tissue engineering, use of the left or right coronary artery would better recapitulate the microenvironment of vascular cells that an implanted cardiac tissue would be interacting with. Co-culture of aortic rings with vEHMs would provide an additional suite of further analyses in host-implant interactions such as in CM-EC cross-talk. ECM remodeling such as by cardiac fibroblasts in vEHMs would provide insight into how implant-mediated remodeling cues influence vascular chemotaxis, such as by creating “highways” of oriented fibrils for directed migration towards vEHMs.

The inventors' work combined the use of three cell types of interest for the applications in cardiac tissue engineering to create complex and robust engineered human myocardial tissues. In-house derivation provides more flexibility in using specific cellular sub-types. Recent literature showed the ability to differentiate mature compact ventricular myocardium, which is associated with the outer ventricular wall for efficient contractile function, as opposed to trabecular myocardium associated with the inner lumen. Coronary artery endothelial cells would be a proper cell type to populate the arteriole-scale channels, as EC sub-types show differing angiogenic properties.

Using hypoimmune hiPSC-derived cells should be useful in clinical interventions where personalized autologous therapeutics are not possible. Pre-clinical work in immune-competent allogeneic mice recipients already showed rescued perfusion in a hind limb ischemia model with implanted hypoimmune hiPSC-endothelial cells and alleviation of functional parameters in infarcted mice hearts with injected hypoimmune hiPSC-cardiomyocytes and hiPSC-endothelial cells.

The in vitro media needs of distinct cell types when in heterocellular culture is challenging considering the current research emphasis on metabolic maturation of cardiomyocytes to push a more adult-like phenotype, primarily by relying on fatty acids to shift the hiPSC-CM metabolic profile towards beta oxidation of lipids. hiPSC-CM structure and metabolism may shift if only transiently exposed to this maturation media. The effects of this media on other key cell types include endothelial cells and fibroblasts.

In advancing vascularization techniques for hiPSC-CM-based therapeutics towards clinical translation, scaling up tissues to sizes appropriate for large animal and eventually clinical trials remains a challenge. Querdel et al. proposed a therapeutic threshold for improving cardiac function to be about 3.3×10⁶ CMs engrafted per gram of heart, or effectively 1×10⁹ cells for an average 300 g human heart. current research suggests average engraftment rates of 10-20% of infarct area, suggesting significant acute hiPSC-CM death post-implantation which can currently only be overcome by delivering far more cells than the target threshold. This greatly motivates work in vascularization as patch sizes to accommodate such a high number of hiPSC-cardiomyocytes necessitates solutions to further mitigate hypoxic stress, particularly once implanted.

RNASeq can be used to better understand exact mechanisms of implanted cardiac tissue remodeling and maturation in vivo. Transcriptional RNASeq data can be used to better understand key factors dictating in vivo cardiac maturation, and particularly any interplay with vascular cross-talk. These data could be used to determine appropriate maturation pathways to upregulate in vitro before implantation such as through CRISPR-Cas9-mediated gene insertion or peptide/small molecule treatments. These analyses combined with epigenetic profiling via ATAC sequencing allow for distinct cell subtype specification in the implant and heart and visualization of population shifts across the initial inosculation stages post-implantation and subsequent remodeling across months.

The inventors also showed the effects of perfusion on reducing implant vessel tortuosity. Exploratory RNASeq should be beneficial in determining exact mechanisms for inducing or otherwise maintaining vascular organization. On an organ level, 3D echocardiographic strain analysis should be performed to understand regional mechanics in the implant and underlying infarct/myocardium, and how vascular tortuosity and perfusion functions in remodeling, vascular and extracellular matrix alignment, and pumping function overtime.

Temporal considerations can be used better understand how implants remodel and persist at longer timepoints. The two week analysis showed more robust engraftment of hiPSC-CMs when perfused in vitro, but studies out to four weeks or greater must be performed to understand if long-term engraftment is maintained, and importantly the impact of prevascularization on maintenance of the implant.

### Example 3

Subcutaneous Implantation of Patterned Vessels to Assess Early Inosculation with Host Vasculature.

Methods: Patterned vessels are fabricated in collagen I gels as described. See Kant, Bare, & Coulombe, Tissue Eng. Part A, 27 (19-20), 1290-1304 (October 2021). Alginate fibers are formed by extruding 1% w/v alginate solution in DI water through a crosslinking bath of 100 mM calcium chloride and collected on a mandrel. Three dried fibers are aseptically embedded into an autoclaved PDMS mold. See Munarin, Kaiser, Kim, Choi, & Coulombe, Tissue Eng. Part C. Methods. 23, 311-321 (2017) and Kaiser, Munarin, & Coulombe, J. Vis. Exp., 2018, 1-7 (2018). Fibers are encapsulated in 500 μL of 2 mg/ml collagen I gel and allowed to set before treating with 1.5 mM sodium citrate for three hours to un-crosslink fibers, resulting in three parallel and patent channels 400 μm in diameter on average. Media is aspirated, then NucRed-transfected human umbilical vein endothelial cells are seeded via injection (25M/mL) into the patent channels. Plates are inverted every twenty minutes for one hour to help with circumferential seeding, then fresh EGM-2 media is pipetted over the gels and incubated overnight.

After four days of in vitro culture, acellular channels (control) or patterned vessels are implanted in vivo. Male RNU Sprague Dawley rats (200-250 g) aged eight weeks are anesthetized with 2-3% isoflurane, then four dorsal subcutaneous pockets are exposed. Control tissues or patterned vessels are sutured in place then the tissue flap is sutured closed. After one day, tissues are explanted and fixed in 4% paraformaldehyde for downstream histological analysis.

Results: At explant, blood flow was apparent in one patterned vessel of an explanted tissue. Histological staining for human-specific CD31 showed possible endothelial cells present in channels. Both acellular channels and patterned vessels showed significant host infiltrate as indicated by hematoxylin and eosin staining, likely an acute immune response to implantation. Blood clots were clear in both control channels and endothelialized vessels, which may suggest initial inosculation that was then blocked by thrombosis. See White et al., Tissue Eng. Part A, 20, 2316-2328 (2014).

### Example 4

### Application of Patterned Vessels as a Therapeutic for Peripheral Artery Disease

Introduction: Peripheral artery disease remains a common and global issue, affecting over 236 million adults world-wide. See Aday & Matsuhita., Circ. Res., 128:1818-1832 (June 2021). Atherosclerotic ischemia and sudden thrombosis can lead to downstream tissue damage and significant functional loss, necessitating interventions to restore blood flow to mitigate long-term impairment. See Campia et al., Am J Med, 132 (10), 1133-1141 (October 2019). Applying patterned vasculature created using biomanufacturing techniques can be used to achieve such goals. Further, this work enables greater understanding of the timeline and mechanism of patterned vessel inosculation in a way that cannot be achieved in deep tissues such as the heart.

Methods: Patterned vessels are fabricated via sacrificial patterning and perfused as previously described. See Kant, Bare, & Coulombe, Tissue Eng. Part A, 27 (19-20), 1290-1304 (October 2021) and Kant et al, Cells, 12 (13), 1698 (June 2023). After five days of in vitro culture, patterned vessels are implanted in vivo in a mouse model of hind limb ischemia. Male C57BL/6J mice aged ten-twelve weeks are anesthetized under 2-3% isoflurane. The femoral artery of one limb is ligated below the inguinal ligament and distal to the superficial epigastric artery, about 5 mm apart. the branches and the femoral artery segment within the two ligations are excised. Control acellular implants, patterned vessels without perfusion, or in vitro-perfused patterned vessels are then sutured into the space between primary ligations, either parallel or perpendicular to blood flow, then the limb is sutured closed.

The ischemic limb and control limbs undergo laser doppler imaging under 2% isoflurane to measure blood flow velocity and track inosculation of the patterned vessels. Blood flow is measured before ligation, after initial surgery, and at three, seven, fourteen, and twenty-one days post-surgery. After twenty-one days, animals are sacrificed following fluorescent lectin injection, and explanted tissues are collected for histological assessment.

### OTHER EMBODIMENTS

Specific compositions and methods of the invention have been described. The detailed description in this specification is illustrative and not restrictive or exhaustive. The detailed description does not intend to limit the disclosure to the precise form described. Other equivalents and modifications besides those already described are possible without departing from the inventive concepts described in this specification, as persons having ordinary skill in the biomedical art will recognize. When the specification or claims recite method steps or functions in an order, alternative embodiments may perform the functions in a different order or concurrently. The inventive subject matter should not be restricted except in the spirit of the disclosure.

All terms should be interpreted in the broadest possible manner consistent with the context when interpreting the disclosure. Unless otherwise defined, the technical and scientific terms used in this specification have the same meaning as commonly understood by persons having ordinary skill in the biomedical art. This invention is not limited to the particular methods, protocols, and reagents described in this specification and can vary in practice. The invention is defined only by the claims.

When a range of values is provided, each intervening value, to the tenth of the unit of the lower limit, unless the context dictates otherwise, between the upper and lower limit of that range and any other stated or intervening value in that range of values.

Some embodiments of the technology described can be defined according to the following numbered paragraphs:

### REFERENCES

Persons having ordinary skill in the biomedical art can rely on these patents, patent applications, scientific books, and scientific publications for enabling methods:

### Patent Citations:

International patent publication WO 2020,232436 (Brown University, Rhode Island Hospital), A human in vitro cardiotoxicity model, published Nov. 19, 2020.
United States patent publication US 2020/0215228 (Coulombe et al.), Collagen microfiber scaffolds, published Jul. 9, 2020.
United States patent publication US 2022/0163511 (Choi & Coulombe), Human in vitro cardiotoxicity model, published May 26, 2022.
United States patent publication US 2022/0268760 (Coulombe et al.), Atrial cardiac microtissues for chamber-specific arrhythmogenic toxicity responses, published Aug. 25, 2022.
United States patent publication US 2023/0020862 (Coulombe et al.), Proangiogenic protein cocktails delivered in custom biomaterials to revascularize ischemic tissue, published Jan. 19, 2023.

### Nonpatent Citations:

Abilez et al., Passive stretch induces structural and functional maturation of engineered heart muscle as predicted by computational modeling. Stem Cells, 36, 265-277 (2018).
Aday & Matsushita, Epidemiology of peripheral artery disease and polyvascular disease. Circulation Research, 128, 1818-1832 (2021).
Afgan et al., The Galaxy platform for accessible, reproducible and collaborative biomedical analyses: 2018 update. Nucleic Acids Res., 46, W537-W544 (2018).
Anbazhakan et al., Blood flow modeling shows improved collateral artery performance during the regenerative period in mammalian hearts, Nature Cardiovasc. Res. 1 (2022) 775-790.
Ballermann, Dardik, Eng, & Liu, Shear stress and the endothelium, Kidney Int., 54, S100-S108 (1998).
Baranski et al., Geometric control of vascular networks to enhance engineered tissue integration and function. Proc. Natl. Acad. Sci. 110, 7586-7591 (2013).
Ben-Shaul, Landau, Merdler, & Levenberg, Mature vessel networks in engineered tissue promote graft—host anastomosis and prevent graft thrombosis, Proc. Natl. Acad. Sci., 201814238 (2019).
Biagi et al., In situ maturated early-stage human-induced pluripotent stem cell-derived cardiomyocytes improve cardiac function by enhancing segmental contraction in infarcted rats, J. Pers. Med., 11, 374 (2021).
Bolger, Lohse, & Usadel, Trimmomatic: a flexible trimmer for Illumine sequence data, Bioinformatics. 30, 2114-2120 (2014).
Brady et al. Guided vascularization in the rat heart leads to transient vessel patterning. APL Bioeng, 4(1) (2020). The Stevens group described the concept of OCT angiograms of implanted vascularized engineered cardiac tissues, although with minimal quantification, low resolution, and a different process of vessel fabrication. In vitro perfusion was not performed. In vivo inosculation of larger vessels was not demonstrated.
Brady et al., Engineered tissue vascularization and engraftment depends on host model. Sci. Rep. 13, 1973 (2023).
Brady et al., Guided vascularization in the rat heart leads to transient vessel patterning. APL Bioeng. 4, 016105 (2020).
Buikema et al., Wnt activation and reduced cell-cell contact synergistically induce massive expansion of functional human iPSC-derived cardiomyocytes, Cell Stem Cell, 27, 50-63.e5 (2020).
Burridge et al., Chemically defined generation of human cardiomyocytes. Nature Methods, 11, 855-860 (2014).
Campia, Gerhard-Herman, Piazza, & Goldhaber, Peripheral artery disease: Past, present, and future. The American Journal of Medicine, 132 (10), 1133-1141 (October 2019).
Cruz-Moreira et al., Assessing the influence of perfusion on cardiac microtissue maturation: A heart-on-chip platform embedding peristaltic pump capabilities, Biotechnol. Bioeng., 118, 3128-3137 (2021).
Debbi et al., Integrating engineered macro vessels with self-assembled capillaries in 3D implantable tissue for promoting vascular integration in-vivo. Biomaterials, 280, 121286 (2022).
DeBenedittis et al., Coupled myovascular expansion directs cardiac growth and regeneration. Development, 149 (2022).
Dhahri et al., In vitro matured human pluripotent stem cell-derived cardiomyocytes form grafts with enhanced structure and function in injured hearts. Circulation (2022).
Dhawan et al., Shear stress and plaque development, Expert Rev. Cardiovasc. Ther., 8, 545-556 (2010).
Dvir et al., Prevascularization of cardiac patch on the omentum improves its therapeutic outcome, Proc. Natl. Acad. Sci., U.S.A., 106, 14990-14995 (2009).
Dwyer, Kant, & Coulombe, Multi-species bulk RNA sequencing on explanted hiPSC-derived engineered myocardium. Galaxy Project.
Eschenhagen, Ridders, & Weinberger, How to repair a broken heart with pluripotent stem cell-derived cardiomyocytes, J. Mol. Cell. Cardiol., 163, 106-117 (2022).
Fang et al., Optimizing bifurcated channels within an anisotropic scaffold for engineering vascularized oriented tissues. Adv. Healthc. Mater., 9, 1-9 (2020).
Feyen et al., Metabolic maturation media improve physiological function of human iPSC-derived cardiomyocytes. Cell Rep., 32, 107925 (2020).
Funakoshi et al., Generation of mature compact ventricular cardiomyocytes from human pluripotent stem cells, Nature Commun. 12 (2021).
Gerbin, Yang, Murry, & Coulombe, Enhanced electrical integration of engineered human myocardium via intramyocardial versus epicardial delivery in infarcted rat hearts. PLoS One, 10, 1-20 (2015).
Gomez-Garcia, Quesnel, Al-attar, Laskary, & Laflamme, Maturation of human pluripotent stem cell derived cardiomyocytes in vitro and in vivo, Semin. Cell Dev. Biol. (2021).
Guo & Pu, Cardiomyocyte maturation, Circ. Res. 126 (2020) 1086-1106.
Helle, Ampuja, Antola, & Kivela, Flow-induced transcriptomic remodeling of endothelial cells derived from human induced pluripotent stem cells, Front. Physiol., 11, 1-13 (2020).
Jiang et al., Construction of chitosan scaffolds with controllable microchannel for tissue engineering and regenerative medicine. Mater. Sci. Eng. C., 126, 112178 (2021).
Jiang et al., Vessel connectivity using Murray's hypothesis, in: J. Invest. Dermatol. (2011), pp. 528-536.
Kaiser, Bellows, Kant, & Coulombe, digital design and automated fabrication of bespoke collagen microfiber scaffolds, Tissue Eng. Part C Methods. 25 (2019) 687-700.
Kaiser, Munarin, & Coulombe, Custom engineered tissue culture molds from laser-etched masters, J. Vis. Exp., 2018, 1-7 (2018).
Kant & Coulombe, Integrated approaches to spatiotemporally directing angiogenesis in host and engineered tissues, Acta Biomater. 69, 42-62 (March 2018).
Kant et al., Patterned arteriole-scale vessels enhance engraftment, perfusion, and vessel branching hierarchy of engineered human myocardium for heart regeneration. Cells, 12(13), 1698 (Jun. 23, 2023).
Kant, Bare, & Coulombe, Tissues with patterned vessels or protein release induce vascular chemotaxis in an in vitro platform, Tissue Eng. Part A, 27 (19-20), 1290-1304 (October 2021).
Kant, Dwyer, Polucha, & Coulombe, Patterned vessels in hiPSC-derived engineered myocardium enhance perfusion and heart regeneration in a rat myocardial infarction model. in Circulation Research, Vol. 131 (August 2022).
Kant, Ph.D. Thesis Dissertation: Patterned arteriole-scale vessels enhance engraftment, perfusion, and maturation of engineered human myocardium for heart regeneration, Brown University, Providence RI, USA, Apr. 25, 2022
Karra et al., Vegfaa instructs cardiac muscle hyperplasia in adult zebrafish, Proc. Natl. Acad. Sci., 115, 8805-8810 (2018).
Kinstlinger et al., Perfusion and endothelialization of engineered tissues with patterned vascular networks. Nature Protoc., 16, 3089-3113 (2021).
Kluin et al., XenofilteR: computational deconvolution of mouse and human reads in tumor xenograft sequence data. BMC Bioinformatics. 19, 366 (2018).
Koffler et al., Improved vascular organization enhances functional integration of engineered skeletal muscle grafts, Proc. Natl. Acad. Sci., U.S.A., 109, 1353-1353 (2012).
Kurokawa et al., Human induced pluripotent stem cell-derived endothelial cells for three-dimensional microphysiological systems. Tissue Eng. Part C Methods., 23, 474-484 (2017).
Laflamme & Murry, Heart regeneration. Nature, 473, 326-335 (2011).
Laflamme et al., Cardiomyocytes derived from human embryonic stem cells in pro-survival factors enhance function of infarcted rat hearts, Nature Biotechnol. 25 (2007) 1015-1024.
Laschke, Vollmer, & Menger, Inosculation: Connecting the life-sustaining pipelines. Tissue Eng. Part B Rev., 15, 455-465 (2009).
Lee, Wu, Jiang, Zhu, & Boas, Dynamic light scattering optical coherence tomography. Opt. Express. 20, 22262 (2012).
Lian et al., Directed cardiomyocyte differentiation from human pluripotent stem cells by modulating Wnt/6-catenin signaling under fully defined conditions. Nature Protoc., 8, 162-175 (2012).
McLellan et al., High-resolution transcriptomic profiling of the heart during chronic stress shows cellular drivers of cardiac fibrosis and hypertrophy. Circulation, 142, 1448-1463 (2020).
Merz et al., Contemporaneous 3D characterization of acute and chronic myocardial I/R injury and response, Nature Commun. 10 (2019) 1-14.
Miki et al., ERRγ enhances cardiac maturation with T-tubule formation in human iPSC-derived cardiomyocytes. Nature Commun., 12, 3596 (2021).
Minor & Coulombe, Stimulating calcium handling in hiPSC-derived engineered cardiac tissues enhances force production, Stem Cells Transl. Med., 1-10 (2022).
Mirabella et al., 3D-printed vascular networks direct therapeutic angiogenesis in ischaemia. Nature Biomed. Eng., 1, 0083 (2017).
Mirdamadi et al., FRESH 3D bioprinting a full-size model of the human heart. ACS Biomater. Sci. Eng., 6, 6453-6459 (2020).
Munarin, Kaiser, Kim, Choi, & Coulombe, Laser-etched designs for molding hydrogel-based engineered tissues., Tissue Eng. Part C. Methods. 23, 311-321 (2017).
Munarin, Kant, Rupert, Khoo, & Coulombe, Engineered human myocardium with local release of angiogenic proteins improves vascularization and cardiac function in injured rat hearts. Biomaterials, 251, 120033 (2020).
Nishimura et al. Formation of vessel-like channel using alginate fiber as a sacrificial structure. Proc. IEEE Int. Conf. Micro Electro Mech. Syst. (MEMS) (2017).
Ogle et al., Distilling complexity to advance cardiac tissue engineering, Sci. Transl. Med. 8, 342ps13-342ps13 (2016).
Parkhideh et al., Perfusable cell-laden matrices to guide patterning of vascularization in vivo. Biomater. Sci. (2023).
Pedde et al., Emerging biofabrication strategies for engineering complex tissue constructs, Adv. Mater. 29, 1-27 (2017).
Peters et al., Metabolic maturation increases susceptibility to hypoxia-induced damage in human iPSC-derived cardiomyocytes. Stem Cells Transl. Med., 11, 1040-1051 (2022).
Qin et al., Depth-resolved 3D visualization of coronary microvasculature with optical microangiography, Phys. Med. Biol. 61 (2016) 7536-7550.
Quaife-Ryan et al., Multicellular transcriptional analysis of mammalian heart regeneration. Circulation, 136, 1123-1139 (2017).
Querdel et al., Human engineered heart tissue patches remuscularize the injured heart in a dose-dependent manner. Circulation, 143, 1991-2006 (2021).
Redd et al., Patterned human microvascular grafts enable rapid vascularization and increase perfusion in infarcted rat hearts. Nat Comm., 10(1), 1-14 (2019). The Murry group demonstrated the use optical coherence tomography (OCT) angiography to image cardiac vasculature and vascularized implants as proof of concept. This analysis was not performed in tissues containing human cardiomyocytes.
Redenski, et al., Microcomputed tomography-based analysis of neovascularization within bioengineered vascularized tissues. ACS Biomater. Sci. Eng., 8, 232-241 (2022).
Riegler et al., et al., Human engineered heart muscles engraft and survive long term in a rodent myocardial infarction model, Circ. Res. 117, 720-730 (2015).
Rottbauer et al., VEGF-PLCγ1 pathway controls cardiac contractility in the embryonic heart, Genes Dev. 19 (2005) 1624-1634.
Rupert & Coulombe, IGF1 and NRG1 enhance proliferation, metabolic maturity, and the force-frequency response in hESC-derived engineered cardiac tissues, Stem Cells Int. 2017 (2017).
Rupert, Irofuala, & Coulombe, Practical adoption of state-of-the-art hiPSC-cardiomyocyte differentiation techniques. PLoS One, 15, e0230001 (2020).
Rupert, Kim, Choi, & Coulombe, Human cardiac fibroblast number and activation state modulate electromechanical function of hiPSC-cardiomyocytes in engineered myocardium, Stem Cells Int. 2020 (2020).
Schaefer et al., A cardiac patch from aligned microvessel and cardiomyocyte patches. J. Tissue Eng. Regen. Med., 12, 546-556 (2018).
Shadrin et al., Cardiopatch platform enables maturation and scale-up of human pluripotent stem cell-derived engineered heart tissues, Nature Commun., 8, 1825 (2017).
Shi et al., Peptide Lv augments L-type voltage-gated calcium channels through vascular endothelial growth factor receptor 2 (VEGFR2) signaling, Biochim. Biophys. Acta—Mol. Cell Res. 1853 (2015) 1154-1164.
Sun & Nunes, Maturation of human stem cell-derived cardiomyocytes in biowires using electrical stimulation. J. Vis. Exp., 2017, 1-8 (2017).
Sun et al., Transplanted microvessels improve pluripotent stem cell-derived cardiomyocyte engraftment and cardiac function after infarction in rats. Sci. Transl. Med. 12, 1-11 (2020).
Szklanny et al., 3D bioprinting of engineered tissue flaps with hierarchical vessel networks (VesselNet) for direct host-to-implant perfusion, Adv. Mater. 33, 2102661 (2021).
Tang et al., Microchannels are an architectural cue that promotes integration and vascularization of silk biomaterials in vivo. ACS Biomater. Sci. Eng., 6, 1476-1486 (2020).
Tiburcy et al., Defined engineered human myocardium with advanced maturation for applications in heart failure modeling and repair. Circulation, 135, 1832-1847 (2017).
Tohyama et al., Distinct metabolic flow enables large-scale purification of mouse and human pluripotent stem cell-derived cardiomyocytes. Cell Stem Cell, 12, 127-137 (2013).
Updegrove et al., SimVascular: An open source pipeline for cardiovascular simulation, Ann. Biomed. Eng., 45, 525-541 (2017).
Vinegoni, Aguirre, Lee, & Weissleder, Imaging the beating heart in the mouse using intravital microscopy techniques, Nature Protoc. 10 (2015) 1802-1819.
Vollert et al., In vitro perfusion of engineered heart tissue through endothelialized channels, Tissue Eng. Part A, 20, 854-863 (2014) demonstrated the technique in the context of engineered cardiac tissues in 2014 as well as perfusion using a bioreactor but did not demonstrate channel lining with endothelial cells or any in vivo feasibility.
Wang et al., Single-cell analysis of murine fibroblasts identifies neonatal to adult switching that regulates cardiomyocyte maturation, Nature Commun. 11, 2585 (2020).
Weinberger et al., Cardiac repair in guinea pigs with human engineered heart tissue from induced pluripotent stem cells. Sci. Transl. Med., 8, 363ra148-363ra148 (2016).
Weyers et al., Effects of cell grafting on coronary remodeling after myocardial infarction. J. Am. Heart Assoc., 2, 1-13 (2013).
Weyers et al., Sonic Hedgehog upregulation does not enhance the survival and engraftment of stem cell-derived cardiomyocytes in infarcted hearts. PLoS One 15(1), e0227780 (2020). The Murry group showed the use of MicroFil in reconstructing the coronary vasculature of the rodent heart and implanted avascular cardiac tissue.
White et al., Implanted cell-dense prevascularized tissues develop functional vasculature that supports reoxygenation after thrombosis, Tissue Eng. Part A, 2316-2328 (2014).

### Textbooks and Technical References

Current Protocols in Immunology (CPI) (2003). John E. Coligan, ADA M Kruisbeek, David H Margulies, Ethan M Shevach, Warren Strobe, (eds.) John Wiley and Sons, Inc. (ISBN 0471142735, 9780471142737).
Current Protocols in Molecular Biology (CPMB), (2014). Frederick M. Ausubel (ed.), John Wiley and Sons (ISBN 047150338X, 9780471503385).
Current Protocols in Protein Science (CPPS), (2005). John E. Coligan (ed.), John Wiley and Sons, Inc.
Immunology (2006). Werner Luttmann, published by Elsevier.
Janeway's Immunobiology, (2014). Kenneth Murphy, Allan Mowat, Casey Weaver (eds.), Taylor & Francis Limited, (ISBN 0815345305, 9780815345305).
Laboratory Methods in Enzymology: DNA, (2013). Jon Lorsch (ed.) Elsevier (ISBN 0124199542).
Lewin's Genes XI, (2014). published by Jones & Bartlett Publishers (ISBN-1449659055).
Molecular Biology and Biotechnology: a Comprehensive Desk Reference, (1995). Robert A. Meyers (ed.), published by VCH Publishers, Inc. (ISBN 1-56081-569-8).
Molecular Cloning: A Laboratory Manual, 4th ed., Michael Richard Green and Joseph Sambrook, (2012). Cold Spring Harbor Laboratory Press, Cold Spring Harbor, N.Y., USA (ISBN 1936113414).
The Encyclopedia of Molecular Cell Biology and Molecular Medicine, Robert S. Porter et al., (eds.), published by Blackwell Science Ltd., 1999-2012 (ISBN 9783527600908).
The Merck Manual of Diagnosis and Therapy, 19th edition (Merck Sharp & Dohme Corp., 2018).
Pharmaceutical Sciences 23^{rd} edition (Elsevier, 2020).

All patents and publications cited throughout this specification are incorporated by reference to disclose and describe the materials and methods that might be used with the technologies described in this specification. The publications discussed are provided only for their disclosure before the filing date. They should not be construed as an admission that the inventors may not antedate such disclosure under prior invention or for any other reason. If there is an apparent discrepancy between a prior patent or publication and the description provided in this specification, the specification (including any definitions) and claims shall control. the statements about the date or contents of these documents are based on the information available to the applicants. These statements constitute no admission to the correctness of the dates or contents of these documents. The publication dates provided in this specification may differ from the actual publication dates. If there is an apparent discrepancy between a publication date provided in this specification and the actual publication date supplied by the publisher, the actual publication date shall control.

1. A dual remuscularization-revascularization system comprising:
(a) vascularized engineered human myocardial tissues (vEHMs) embedded in a biomaterial matrix,
wherein the embedded vEHMs are in a pattern,
wherein the embedded vEHMs mid-diameter vessels; and
(b) endothelial cells coated on the vessel structure,
wherein the endothelial cells are localized to a vessel wall.

2. The system of claim 1, wherein the components of the system are biocompatible.

3. The system of claim 1, wherein the components of the system are degradable.

4. The system of claim 1, wherein the system is within a packaging.

5. The system of claim 1, wherein the vEHMs have a high cardiomyocyte density.

6. The system of claim 1, wherein the embedded vEHMs comprise both mid-diameter vessels and large diameter vessels.

7. The system of claim 1, wherein the embedded vEHMs comprise vessels that are branched.

8. The system of claim 1, wherein the vessels perfuse the whole of the embedded vEHMs.

9. The system of claim 1, wherein the vessels are patterned for the largest inlet vessel to be micro-surgically anastomosed to a host vessel.

10. The system of claim 1, wherein the vessels have been self-connected to a subject's vasculature.

11. The system of claim 1, wherein the patterned, arteriole-scale vasculature successfully is inosculated with the vasculature of a subject.

12. The system of claim 1, wherein such implanted tissues are used in surgical settings to restore cardiac vascular or contractile function for patients with ischemic heart disease.

13. A method of making a dual remuscularization-revascularization system comprising vascularized engineered human myocardial tissues (vEHMs), comprising the steps of:
(a) mixing hiPSC-cardiomyocytes and human primary cardiac fibroblasts into a biomaterial matrix;
(b) embedding fibers coated with endothelial cells in the biomaterial matrix to form a pattern with arteriole-scale channels; and
(c) culturing the vascularized engineered human myocardial tissues under dynamic perfusion conditions.

14. A method of making a dual remuscularization-revascularization system comprising vascularized engineered human myocardial tissues (vEHMs), comprising the steps of:
(a) embedding fibers coated with endothelial cells in a biomaterial matrix to form a pattern with arteriole-scale channels;
(b) perfusing hiPSC-cardiomyocytes into the biomaterial matrix in vitro;
(c) culturing the vascularized engineered human myocardial tissues under dynamic perfusion conditions.
